(19) United States
(12) Patent Application Publication
(10) Pub. No.: US 2025/0066729 A1
(43) Pub. Date: Feb. 27, 2025

(54) Systems and Methods Incorporating Modified T-Cells

(71) Applicant: The Board of Trustees of the Leland Stanford Junior University, Stanford, CA (US)

(72) Inventors: Mark M. Davis, Atherton, CA (US); Xin Chen, Mountain View, CA (US); Mustafa Ghanizada, Copenhagen (DK); Venkata Vamsee Aditya Mallajosyula, East Palo Alto, CA (US)

(21) Appl. No.: 18/726,384

(22) PCT Filed: Jan. 9, 2023
(86) PCT No.: PCT/US2023/010431

§ 371 Date: Jul. 2, 2024

Related U.S. Application Data
(60) Provisional application No. 63/298,104, filed on Jan. 10, 2022.

(51) Int. Cl. C12N 5/0783; C07K 16/10; C07K 16/40; C12N 5/078; C12N 5/0781; C12N 5/0784

(52) U.S. Cl. CPC C12N 5/0637; C07K 16/1002; C07K 16/1018; C07K 16/1027; C07K 16/40; C12N 5/0635; C12N 5/0639; C12N 5/0651; C12N 2501/25; C12N 2501/599; C12N 2510/02; C12N 2513/00

(57) ABSTRACT

Disclosed herein are methods, systems and devices to model adaptive immune responses and develop and/or test improved antibodies, vaccines, and other therapeutic agents. The adaptive immune responses can be modeled using lymphoid tissue derived from a subject. The methods, systems and devices disclosed herein can comprise modified T-cells.

### CROSS REFERENCE TO OTHER APPLICATIONS

This application claims the benefit of U.S. Provisional Application No. 63/298,104 filed Jan. 10, 2022, the contents of which are hereby incorporated by reference in its entirety.

### STATEMENT AS TO FEDERALLY SPONSORED RESEARCH

This invention was made with government support under grant number U19-AI057229 awarded by the National Institute of Allergy and Infectious Diseases and the Howard Hughes Medical Institute. The government has certain rights in the invention.

### BACKGROUND

Antigen recognition by lymphocytes has been studied by immunologists since the discovery of antibodies and their specificities over a century ago, followed by the more recent discovery of T cells and their antigen receptors in the 1960s-1980s. The B cells that are responsible for forming a neutralizing antibody response develop with germinal centers (GCs) and extrafollicular regions in lymphoid organs. Upon antigen presentation by antigen-presenting cells (APCs), T follicular helper (TFH) cells, and a variety of hematopoietic and non-hematopoietic cells interact and deliver signals to GC B cells for survival, proliferation, antibody affinity maturation, class-switch recombination and differentiations. Almost all of these interactions have been elucidated through in vivo studies of inbred mice. While these have produced a wealth of important information, the lack of a system that replicates the essential features of adaptive immunity, such as affinity maturation and class switching, and the effects of adjuvants, leaves many mechanistic aspects inaccessible.

Much of what is known about adaptive immunity has come from inbred mouse studies, using methods that are often difficult or impossible to confirm in humans. Often vaccine responses in mice are poorly predictive of responses to those same vaccines in humans. The present invention uses cells from lymphoid organs to develop a functional organotypic system that recapitulates key features germinal centers and adaptive immunity in vitro, including but not limited to the production of antigen-specific antibodies, somatic hypermutations, affinity maturation, plasmablast differentiation and class-switch recombination.

Many methods of antibody production involve immunization of antigen to nonhuman animals, generation of hybridoma cell lines, collection, and purification of the antibody. A challenge of these methods is the production of a human glycosylation pattern in antibodies produced from nonhuman subjects. Non-human glycosylation patterns can elicit an immune response in humans.

The methods and systems disclosed herein utilize cells from human lymphoid organs to produce human antibodies in response to antigens. Regulatory systems of the immune system, comprising regulatory T-cells, prevent the production of antibodies to antigens produced by the body, e.g., self-antigens. The invention disclosed herein provides advantageous system to produce high affinity antibodies to therapeutic targets including self-antigens.

### SUMMARY

Disclosed herein are methods, systems, and devices capable of producing human antibodies to antigens, including self-antigens. Disclosed herein are methods, systems and devices capable of modeling adaptive immune responses. Disclosed herein is an in vitro cell cluster comprising lymphoid cells, wherein said in vitro cell cluster is derived from lymphoid tissue of a subject, said in vitro cell cluster comprising a germinal center configured to produce antibodies to an antigen, wherein said germinal center comprises genetically modified T-cells. The antigen can be a self-antigen. The antigen can be a polysaccharide, lipid, nucleic acid, peptide, protein or fragment thereof. The protein can be a viral protein, a growth factor, a cancer related protein, or an auto-immune disease related protein. The antigen can be expressed by a tissue of said subject. The antigen can be a vaccine or vaccine candidate.

The germinal center can comprise genetically modified T-cells. The genetically modified T-cells can be regulatory T-cells. The genetically modified T-cells can be modified to knock down or knock out expression of a forkhead box transcription factor. The forkhead box transcription factor can be FoxP3. The genetically modified T-cells can be modified to knock down or knock out expression of granzyme B (GZMB). The genetically modified T-cells can be CD8⁺ T-cells. The genetically-modified CD8+ T-cells can be modified to knock down or knock out an expression of granzyme B. The in vitro cell cluster can further comprise one or more adjuvants. The one or more adjuvants can comprise aluminum hydroxide or imiquimod. The germinal center can be configured to perform one or more of: hypermutation maturation, affinity maturation, plasmablast differentiation, and class switching recombination. The cells can be configured to differentiate to form said in vitro cell cluster upon exposure to said antigen.

The antigen can be a peptide, protein or fragment thereof. The protein can be a viral protein, a growth factor, a cancer related protein, or an auto-immune disease related protein.

The lymphoid cells can be derived from tonsil tissue, spleen tissue, adenoid tissue, thymus tissue, or lymph node tissue.

The germinal center can comprise antigen presenting cells (APCs) and T-cells at least partially surrounding said functional germinal center. The APCs can comprise B-cells. The APCs can be professional antigen presenting cells. The APCs can comprise dendritic cells. The dendritic cells can be follicular dendritic cells. The B-cells can comprise CD38+ B-cells. The B-cells can comprise CD27+ B-cells. The T-cells can comprise CD8+ T-cells. The T-cells can comprise CD4+ T-cells. The antibodies can have an affinity to said antigen between 1 nanomolar and 10 femtomolar. The antibodies can have an affinity to said antigen up to 1 femtomolar or 10 femtomolar.

Disclosed herein is a method for generating antibodies from a lymphoid organoid, comprising placing lymphoid cells in a media to produce said lymphoid organoid, wherein said lymphoid organoid comprises T-cells and B-cells, wherein said T-cells are modified to have a decreased regulation of B-cells as compared to unmodified T-cells; introducing an antigen to said media; incubating said lymphoid organoid with said antigen to generate said antibodies; and isolating said antibodies from said lymphoid organoid. The method can further comprise isolating nucleic acids encoding said antibodies from said lymphoid organoid.

The lymphoid cells can be obtained from a subject, wherein said antigen is expressed by a tissue of said subject. The subject can be human. The T-cells can be modified to reduce or eliminate expression of a forkhead box transcription factor. The forkhead box transcription factor can be FoxP3. The T-cells can be modified to reduce or eliminate expression of a granzyme B (GZMB). The T cells can be modified by in vitro programmed genome editing, e.g. with a CRISPR-based system.

The method can further comprise introducing a B-cell activating factor to said media. The method can further comprise introducing an adjuvant to said media. The adjuvant can be aluminum hydroxide or imiquimod. The method can further comprise incubating said lymphoid organoid with said antigen and said adjuvant. The adjuvant can be introduced after said antigen. Incubating can be for at least 48 hours.

The method can further comprise obtaining said lymphoid cells from a subject. The lymphoid cells can be obtained by biopsy. The lymphoid cells can be derived from tonsil tissue, spleen tissue, adenoid tissue, thymus tissue, or lymph node tissue. The antigen can be a polysaccharide, lipid, nucleic acid, peptide, protein, or fragment thereof. The protein can be a growth factor, a cancer related protein, or an auto-immune disease related protein. The antigen can be a vaccine or vaccine candidate. The method can further comprise introducing an immune system stimulant. The immune system stimulant can be a live attenuated influenza virus (LAIV). The LAIV can be introduced with said antigen. The antibodies can have an affinity to the antigen between 1 nanomolar and 10 femtomolar.

Disclosed herein is an in vitro cell cluster comprising lymphoid cells, wherein said in vitro cell cluster is derived from lymphoid tissue of a subject, said in vitro cell cluster comprising a germinal center configured to produce antibodies to a self-antigen, wherein said germinal center comprises genetically modified T-cells. Disclosed herein is a method for generating antibodies from a lymphoid organoid, comprising placing lymphoid cells in a media to produce the lymphoid organoid, wherein the lymphoid organoid comprises T-cells and B-cells, wherein the T-cells are modified to have a decreased regulation of B-cells as compared to unmodified T-cells; introducing a self-antigen to the media; incubating said lymphoid organoid with the self-antigen to generate the antibodies; and isolating the antibodies from the lymphoid organoid.

Disclosed herein is an in vitro cell cluster comprising lymphoid cells, wherein said in vitro cell cluster comprises a germinal center and an aggregate of T-cells; wherein said in vitro cell cluster is configured to maintain said germinal center and said aggregate of T-cells and a cellular respiration for at least 24 hours. The functions of this cluster can be modulated by one or more adjuvants. These can comprise aluminum hydroxide or imiquimod, or any of a number adjuvants in current use or those in development. The germinal center can be configured to perform one or more of: hypermutation maturation, affinity maturation, plasmablast differentiation, class switching recombination, and antigen-specific antibody production. The cells can be configured to differentiate to form said in vitro cell cluster upon exposure to an antigen. The antigen can be a peptide, protein or fragment thereof, polysaccharides, lipids, nucleic acids, or other biomolecules. The protein can be a viral protein, a growth factor, a cancer related protein, or an auto-immune disease related protein. The antigen can be a vaccine or vaccine candidate. The carbohydrate can be a bacterial coat protein or a fragment thereof. Many natural protein antigens, such as influenza hemagglutinin, are glycosylated, and can stimulate protein/peptide specific antibody expressing B cells, or T cells, or these specific lymphocytes can be stimulated by carbohydrates, or glycosylated proteins or peptides in any combination. The lymphoid cells can be derived from tonsil tissue, spleen tissue, adenoid tissue, thymus tissue, or lymph node tissue. The germinal center can comprise antigen presenting cells (APCs) and T-cells at least partially surrounding said functional germinal center. The APCs can comprise B-cells or dendritic cells. The dendritic cells can comprise follicular dendritic cells. The B-cells can comprise CD38+ B-cells. The B-cells can comprise CD27+ B-cells. The T-cells can comprise CD8+ T-cells or CD4+ T-cells of the αβ type; or they can comprise γδ T cells.

Disclosed herein is an in vitro lymphoid culture system comprising a well, the well comprising a cell-suspension of lymphoid cells comprising T-cells, B-cells and non-lymphoid cells that are found in lymphoid organoids and participate in these reactions (Wagar et al 2021), plus media; wherein said media provides nutrients and factors that are needed for proper cell differentiation and the spatial organization of germinal centers and the adjacent aggregate of T-cells. The media can comprise recombinant human B-cell activating factor (BAFF) and IL-2. The media can also comprise one or more adjuvants and a vaccine or vaccine candidate, including, but are not limited to, inactivated pathogen vaccines; live-attenuated pathogen vaccines; messenger RNA (mRNA) vaccines; subunit, recombinant, polysaccharide, and conjugate vaccines; toxoid vaccines; and viral vector vaccine candidates. The one or more adjuvants can comprise aluminum hydroxide or imiquimod, or many other adjuvants in use or being developed. The germinal center can perform one or more of: hypermutation maturation, affinity maturation, plasmablast differentiation, class switching recombination, and antigen-specific antibody production. The cells can be stimulated by introduction of an antigen to said media. The antigen can be a protein or fragment thereof, or a carbohydrate or a glycoprotein or fragment thereof. The protein can be a viral protein, a growth factor, a cancer related protein, or an auto-immune disease related protein. The lymphoid cells can be derived from one or more of tonsil tissue, spleen tissue, adenoid tissue, thymus tissue, or lymph node tissue. The germinal center can comprise antigen-presenting cells (APCs) and T-cells outside of said functional germinal center. The APCs can comprise B-cells or dendritic cells. The dendritic cells can comprise follicular dendritic cells. The B-cells can comprise CD38+ B-cells. The B-cells can comprise CD27+ B-cells. The T-cells can comprise CD8+ T-cells or CD4+ T-cells of the αβ type; T cells can comprise γδ cells. The T cells can be genetically modified to knock down or knock out expression of a forkhead box transcription factor. The T cells can be genetically modified to knock down or knock out expression of granzyme B.

The spatial organization of lymphoid tissue can be maintained for at least four days. The spatial organization of lymphoid tissue can be maintained for at least one week. The spatial organization of lymphoid tissue can be maintained for at least two weeks. The spatial organization of lymphoid tissue can be maintained for at least three weeks.

Disclosed herein is a method for generating antibodies, in some embodiments high affinity antibodies, from a lymphoid organoid, comprising (a) placing lymphoid cells in a media to produce said lymphoid organoid, wherein said lymphoid organoid comprises a spatial organization of lymphoid tissue, said spatial organization comprising a germinal center and an aggregate of T-cells; (b) introducing an antigen to said media; (c) incubating said lymphoid organoid with said antigen to generate said antibodies; and isolating said antibodies from said lymphoid organoid. Step (a) can further comprise introducing a B-cell activating factor to said media. Step (b) can further comprise introducing an adjuvant to said media. The adjuvant can be aluminum hydroxide or imiquimod. Step (c) can further comprise incubating said lymphoid organoid with said antigen and said adjuvant. The adjuvant can be introduced after said antigen. The incubating can be for at least 48 hours. The method can further comprise obtaining said lymphoid cells from a subject. The lymphoid cells can be obtained by biopsy. The lymphoid cells can be derived from tonsil tissue, spleen tissue, adenoid tissue, thymus tissue, or lymph node tissue. The antigen can be a peptide, protein, protein encoded by an mRNA vaccine, or fragment thereof. The protein is a viral protein, a bacterial protein, a growth factor, a cancer related protein, or an auto-immune disease related protein. The virus can be a coronavirus. Coding sequences for antibodies can be identified, and introduced into an expression vector for production of a selected antibody by conventional methods. Antibodies thus produced can be high affinity antibodies.

Disclosed herein is a method of screening a patient for immune response to a treatment comprising obtaining a sample comprising lymphoid cells from a patient; placing said lymphoid cells in a system as described herein; exposing said lymphoid cells to a treatment to stimulate the production of antibodies; isolating said antibodies from said system; and assaying said antibodies. The sample can be selected from one or more of tonsil tissue, spleen tissue, adenoid tissue, thymus tissue, or lymph node tissue. The sample can be obtained by biopsy.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIGS. 1A-1F show the adaptive immune responses in tonsil organoids. FIG. 1A shows a workflow for tissue disruption and culture preparation with representative stereoscope images from day-5 unstimulated or live attenuated influenza vaccine (LAIV)-stimulated replicate cultures. Light regions are areas of high cell density. FIG. 1B shows cell composition of immune cell types in freshly isolated, frozen and revived day-0 tonsil cells, day-7 unstimulated cultures and day-7 LAIV-stimulated cultures. Frequencies were determined by flow cytometry, and plotted values are the mean±s.e.m. (n=5 donors). PB/PCs, plasmablast/plasma cells; NK, natural killer. FIG. 1C shows Plasmablast differentiation and specific antibody secretion from unstimulated and LAIV-stimulated organoids from day-7 cultures—paired, two-sided Wilcoxon signed-rank tests were used to calculate P values (n=15 donors). OD, optical density. FIG. 1D shows antigen-specific antibodies and total IgG from day-7 unstimulated and LAIV-stimulated organoid cultures. Colors represent individual donors (n=6). FIG. 1E shows ASCs specific for influenza antigens from day-7 unstimulated and LAIV-stimulated cultures (n=5 donors). FIG. 1F shows Plasmablast frequencies and antibody secretion on day 14 from immune organoids derived from human lung-draining lymph nodes and spleens (n=2 for each tissue). A/Cal, A/California H1N1 virus.

FIGS. 2A-2F show longitudinal tracking of immune organoids to reveal cellular and functional changes consistent with an adaptive response. FIG. 2A shows representative flow cytometry staining of B cell differentiation phenotypes in unstimulated and LAIV-stimulated cultures-cells shown are pre-gated on total B cells (CD45+CD19+CD3−). FIGS. 2B-2C show quantitation of B cell (b) and T cell (c) phenotypes across time in unstimulated and LAIV-stimulated organoids. FIG. 2D shows influenza-specific antibodies detected in culture supernatants. FIG. 2E shows the viral neutralizing capacity of antibodies in culture supernatants against two of the four vaccine strains represented in the antigen stimulation (LAIV 2015-2016)—complete cellular and humoral data from n=3 donors (a-e) and longitudinal antibody detection data from an additional n=4 donors (d) are shown. FIG. 2F shows influenza M1-specific CD8+ T cell responses in unstimulated and LAIV-stimulated tonsil organoids from four HLA-A2+ donors. Donor ages (in years) are shown in parentheses after donor IDs.

FIGS. 3A-3F show diversity and maturation of the influenza response. FIG. 3A shows confocal microscopy images of a day-4 LAIV-stimulated organoid culture and B cell areas with light zone (LZ) and dark zone (DZ) organization. FIG. 3B shows single-cell RNA-seq of B cells from day-0 tonsil cells and organoid cultures from days 5 and 9 from one donor—the fold change values in genes and antibodies (Ab) highly expressed in GC compared to naive B cells (at least 1.5-fold increase) were plotted from day-0 (d0) B cells and day-5 (d5) LAIV-stimulated B cells. FIG. 3C shows median fluorescence intensity (MFI) for protein-level AID expression (top) and the relative proportions of B cell subsets (bottom) on days 0, 4 and 7 of organoid culture-Box plots show median values with hinges representing the first and third quartiles and whiskers representing the highest and lowest value that is within 1.5 times the interquartile range of the hinges (n=4 donors). FMO, fluorescence minus one. FIG. 3D shows single-cell BCR sequencing of high-affinity A/California 2009 H1N1 HA-specific plasmablasts and GC B cells from one representative donor. High-affinity B cells specific for HA from day-0 (black points) and day-7 LAIV-stimulated organoid cultures (green, blue and red points representing IgM, IgA and IgG isotypes, respectively) are shown-clonal families are represented by open shading. FIG. 3E shows organoid cultures pre-depleted of high-affinity A/California HA+ B cells and non-naive B cells produce new HA+ B cells-effective depletion on day 0 of HA+ B cells and non-naive populations was confirmed by post-sort analysis, and new HA+ B cells were detectable on day 10—data shown are representative plots from one of n=4 donors. FIG. 3F shows influenza-specific antibodies were detected in depleted organoid cultures from three of n=4 donors tested. Donor ages (in years) are shown in parentheses after donor IDs.

FIGS. 4A-4E show somatic hypermutation and antigen-directed affinity maturation supported in tonsil organoids. FIG. 4A shows BCR repertoire analysis workflow on cultures prepared from tonsil cells depleted of high-affinity HA+ and total non-naive B cells (n=6 donors). FIG. 4B shows Influenza vaccine-specific and A/California 2009 H1N1 HA-specific antibodies after 7 d in unstimulated and LAIV-stimulated cultures. FIG. 4C shows Somatic hypermutation as measured by the number of nucleotide mutations from the germline heavy-chain BCR sequences-data are mean±s.e.m. and significance was calculated with two-sided Welch t-tests. FIG. 4D shows clone size (as measured by the number of RNA molecules per lineage) and diversity (as measured by the number of unique RNA molecules per lineage) in A/California 2009 H1N1 HA+ B cell lineages compared to non-A/California HA+ lineages were measured in n=6 donors—specificity for A/California HA was inferred from the pool of known day-0 HA+ BCRs. Box plots show median values with hinges representing the first and third quartiles and whiskers representing the highest and lowest value that is within 1.5 times the interquartile range of the hinges. Each overlaid point represents an individual lineage. Significance values shown were calculated using two-sided Welch t-tests. Donor ages (in years) are shown in parentheses after donor IDs. FIG. 4E shows development of HA specificity in a BCR lineage from one donor. The size of the node represents the number of RNA molecules detected, the distance between nodes is proportional to the edit distance (nucleotide) between them, and the color reflects edit distance (amino acid) to the nearest known A/California HA-specific sequence, with light blue representing an exact match. The root sequence, major node and a subset of the major node with IgG class switch are highlighted.

FIGS. 5A-5H show depletion studies that reveal cell types required for plasmablast differentiation, specific antibody secretion and antibody affinity in organoid cultures. FIGS. 5A-5B show the effect of cell subset depletion on plasmablast differentiation (FIG. 5A) and specific antibody secretion (FIG. 5B). Fold change in plasmablast frequency is relative to sorted cells reconstituted with the depleted cell type. Sort depletion experiments were performed on cells from n=6 (naive B cells and non-naive B cells), n=2 (total B cells and plasmacytoid dendritic cells or pDCs) or n=3 (myeloid cells+pDCs, regulatory T (Treg) cells, memory B cells and plasmablasts) donors. FIGS. 5C-5D show the effect of CD4+ depletion on plasmablast differentiation (FIG. 5C) and antibody production after 7 d in organoid culture (n=15 donors) (FIG. 5D). Antibodies against influenza were not detected in unstimulated controls. Paired, two-sided Wilcoxon signed-rank tests were used to determine the significance values. FIG. 5E shows dependence on CD4+ cells for influenza-specific antibody secretion was associated with age. Intact or CD4+ depleted, LAIV-stimulated cultures were stratified into ages 5 years and under (red) or older than 5 (cyan). Box plots show the median values with hinges representing the first and third quartiles and whiskers representing the highest and lowest value that is within 1.5 times the interquartile range of the hinges. Unpaired, two-sided Mann-Whitney U tests were used to calculate significance. FIG. 5F shows biolayer interferometry binding data from intact or CD4+-depleted cultures. Dissociation rates as measured by kd (1/s) are shown for four donors and detailed binding traces from one representative donor. Data were collected from antibodies binding full-length A/California 2009 H1N1 HA or head domain only. Stem-specific antibodies were detected in two donors but only under CD4-containing conditions. FIG. 5G shows minimal cell requirements to support an influenza B cell response. Individual cell types were sorted and then recombined as shown to test for plasmablast differentiation and antibody secretion after 7 d of stimulation with LAIV (n=7 donors). FIG. 5H shows contribution of naive versus memory CD4+ T cells in B cell differentiation and influenza-specific antibody secretion under minimal cellular conditions. Naive B cells, APCs and CD45− cells were sorted and combined with equal numbers of either naive (CD45RA+CD27+) or memory (all non-naive) CD4+ T cells for 10 d in the presence of LAIV (n=5 donors).

FIGS. 6A-6G show the ability to respond to non-influenza antigens and immunomodulation with adjuvants. FIGS. 6A-6B show plasmablast differentiation in response to MMR vaccine stimulation in tonsil organoid cultures (FIG. 6A) and their corresponding MMR-specific IgG production (FIG. 6B). Organoid cultures from seven donors were harvested on day 11 for flow cytometry analysis and antibody secretion. Significance values were calculated using paired, two-sided Wilcoxon signed-rank tests. Donor ages are shown in parentheses. FIG. 6C shows PE-specific B cells were detected at low levels in unstimulated cultures (0.05% of total B cells) and were increased with PE stimulation but not with an irrelevant antigen (n=3 donors; day 11 after stimulation). FIG. 6D shows PE-specific B cell frequencies were enhanced by stimulation in the presence of alum (n=4 donors; day 10 after stimulation). Representative flow cytometry plots are shown from one donor. Individual points are shown for each donor. Treatment groups were tested with paired, two-sided t-tests to determine significance values. FIG. 6E shows tonsil organoid responses to rabies vaccine, as indicated by pre-GC, GC and plasmablast B cell phenotypes and antigen-specific IgM after 14 d of culture. NP, nucleoprotein. P values between treatment groups were determined with paired, two-sided t-tests (n=10 donors). FIG. 6F shows lung-draining lymph nodes and spleen organoids were stimulated for 14 d with rabies vaccine. FIG. 6G shows tonsil organoids were stimulated with vaccine candidates for SARS-COV-2 (n=12 donors). Ad5 vectors containing either spike protein(S), spike and nucleocapsid protein (SN) or the S1 spike subunit with nucleocapsid (S1N) were used for stimulation, and responses were measured after 14 d in culture. Box plots show the median values with hinges representing the first and third quartiles and whiskers representing the highest and lowest value that is within 1.5 times the interquartile range of the hinges. Culture supernatants were collected and tested for antigen-specific IgM, IgG and IgA. One donor that was positive for multiple proteins is shown. An irrelevant adenoviral vector with norovirus VP1 was used as a control. Paired, two-sided Wilcoxon signed-rank tests were used to calculate P values shown (not adjusted for multiple comparisons).

FIGS. 7A-7D show characteristics of B cells from tonsil organoid cultures. FIG. 7A shows Flow cytometry gating scheme on representative unstimulated and LAIV-stimulated cultures. FIG. 7B shows representative ELISpot wells for detection of Ab-secreting cells with influenza vaccine specificity. The number of spots detected are shown in the corner of each well. FIG. 7C shows a comparison of organoid cultures grown in standard flat −bottom wells vs. transwells. Data shown are from day 7 LAIV-stimulated cultures (n=6 donors). p values shown were determined with paired Wilcoxon signed-rank two-sided tests. Boxplots show median values with hinges representing first and third quartiles and whiskers representing the highest and lowest value that is within 1.5× the interquartile range of the hinges. FIG. 7D shows a correlation between specific Ab secretion and plasmablast frequency in influenza-stimulated and unstimulated cultures on day 7. Detection of influenza-specific IgG antibodies by semi-quantitative indirect ELISA. Optical density was corrected for background using unspent culture medium as a control. Each donor (n=15) was tested under four conditions: no stimulation, IIV-stimulated, LAIV-stimulated, or H1N1 WT virus-stimulated. Plasmablast (CD19+CD38+++CD27+) frequencies were determined by flow cytometry.

FIGS. 8A-8E show confocal imaging showing distribution of different cell types and their interactions in tonsil tissue and organoid cultures. Merge does not include DAPI staining for clarity. FIG. 8A shows one fresh tonsil tissue stained with panels of markers to define I and B cell areas and GC structure. FIG. 8B shows day 4 unstimulated organoid stained for I and B cell markers. FIG. 8C shows day 4 LAIV organoid stained for germinal center markers. FIG. 8D shows day 4 unstimulated organoid stained for B and I cell distribution. FIG. 8E shows day 4 LAIV organoid stained for markers of I follicular helper cells. For organoid cultures (b-e), representative examples are shown from organoid cultures derived from three different donors.

FIGS. 9A-9B show the quantification of organoid organization and function. FIG. 9A shows the quantification of CXCR4 and CD83 expression levels (mean intensity, left panel) and percent positive (percentage of CD20+ B cells, right panel) in day 4 tonsil organoids from one donor. Two areas of an LAIV-stimulated organoid (top GC and bottom GC as also shown in FIG. 3a) and a representative GC-organized area from an unstimulated organoid were used for the calculation. FIG. 9B shows representative intracellular AID flow cytometry staining profiles. Data shown are from a day 4, LAIV-stimulated organoid culture. Total B cells were subsetted based on CD38 and CD27 expression and are shown as individual profiles in red compared to a ‘no AID primary antibody’ FMO control in grey.

FIGS. 10A-10B show B cell receptor sequencing from HA-specific B cells. FIG. 10A shows phylogenetic trees from two donors are shown (top). Day 0 clones are indicated by black points. Different isotypes (IgM, IgA, IgG) are indicated by color (green, blue, and red respectively). Oligoclonal populations and clonal families from single cell data are represented by larger points (open circles). Clonal families were defined as BCR sequences that use the same V and J genes and have at least 70% amino acid identity in the CDR3 region for heavy and light chains. FIG. 10B shows FACS staining and B cell phenotypes of HA-specific B cells compared to the total B cell pool.

FIG. 11 shows tracking of individual heavy-chain BCR lineages before and after LAIV stimulation in tonsil organoids show evidence of isotype switching. Immunoglobulin heavy-chain gene rearrangements for each isotype were sequenced from total memory B cells, germinal center B cells, and plasmablasts sorted from cultures of four donors on days 0 and 7. Heavy-chain BCR sequences from lineages that contained members as only IgM on day 0 and as only isotype-switched on day 7 were compared for their somatic hypermutation levels. For each lineage, the mean SHM was calculated for day 0 IgM members and for day 7 switched members, and the difference between these means was plotted. Lineages with increased mutation are shown in gray and those with decreased mutation in black.

FIG. 12 shows depletion of pre-existing HA-specific and non-naive B cells do not prevent production of new high-affinity HA+ B cells after organoid culture. B cells with high affinity BCRs for A/California 2009 HA+ and all non-naive B cells were depleted by FACS and depletion was confirmed by post-sort analysis. After 10 days in organoid culture, cells were harvested, re-stained for A/California HA+ B cells, and run on a flow cytometer. n=4 donors were tested.

FIGS. 13A-13C shows effects of cell depletion on influenza-specific antibodies and their affinities. FIG. 13A shows that rescue of plasmablast and Ab responses to LAIV stimulation by supplementing pDC-depleted cultures with type I IFNs. FIG. 13B shows the frequency of CD4+ T cells (of live cells) in intact and CD4-depleted cultures on day 7. CD4+ cells were depleted by positive selection with magnetic particles. FIG. 13C shows a biolayer interferometry data indicating Ab affinity for A/California 2009 H1N1 HA full length, head-specific, and stem domains. Colors are matched to patient samples in (b).

FIG. 14 shows SARS-COV2-specific Abs detected in organoid cultures stimulated with Ad5 vectored vaccine candidates. Day 14 post-stimulation culture supernatants were tested for the presence of SARS-COV2-specific IgM, IgG, and IgA in n=12 donors. Abs were detected using a protein microarray. Individual donors are represented by symbols. The signal intensities shown were background subtracted based on unstimulated control cultures from the same donor. Boxplots show median values with hinges representing first and third quartiles and whiskers representing the highest and lowest value that is within 1.5× the interquartile range of the hinges.

FIG. 15 shows PR3 specific autoantibodies produced by tonsil organoids. Populations of T cells were depleted before being stimulated with LAIV or LAIV with self-antigen cocktails (LAIV+A). Results show that upon stimulation with LAIV+A in tonsil organoids with CD4+CD25+ T cell depletion, a large amount of PR3 specific autoantibody was generated as measured by ELISA.

FIG. 16A-16F show FACS results of FOXP3-KO in T cells using CRISPR/Cas9 gene editing kit.

FIG. 17 shows specific autoantibody production against each self-antigen in FOXP3-KO T cells upon LAIV+A stimulation. Four different autoantibodies generated against each self-antigen (proteinase 3 (PR3), double stranded DNA (dsDNA), histone, and SNRNP70) from the self-antigen cocktails were measured using ELISA. Knockout of FOXP3 alone in T cells is sufficient to allow a large amount of specific autoantibody production.

FIG. 18 shows an example of a computer system configured to implement methods provided herein.

FIGS. 19A-19B show frequency of the CD4⁺ and CD8⁺ regulatory T cells in human tonsils and PBMCs. FIG. 19A shows a representative FACS plot showing CD25, FOXP3, and CXCR5 expression of CD4⁺ T cells from tonsils and PBMCs. FIG. 19B shows the percentage of FOXP3⁺ cells, Tfr (CXCR5⁺FOXP3⁺), CD25+FOXP3⁺ cells in CD4⁺ T cells, and KIR+CD8+ T cells from donors (N=7) with PBMC and tonsil samples. The mean±SEM is indicated. ****p<0.0001. *p<0.05 (Unpaired t-test).

FIGS. 20A-20D show inflammatory T and B cell phenotypes in FOXP3 and GZMB KO tonsil organoids. As shown in FIG. 20A, fold change frequency of activated CD4, and CD8 T cells (CD27⁺CD38⁺) in FOXP3 KO and GZMB KO tonsil organoids over control were compared after 10-day culture. Representative data from four independent experiments. The mean±SEM is indicated. *p<0.05 (Unpaired t-test). As shown in FIG. 20B, the frequency of activated T_{FH} cells in control, FOXP3 KO, and GZMB KO tonsil organoids were compared after 10-day culture. Representative data from three independent experiments. The mean±SEM is indicated. **p<0.01 (Paired t-test). FIG. 20C shows representative FACS staining of B cell differentiation phenotypes in control, FOXP3 KO, and GZMB KO tonsil organoids after 10-day culture. Cells were pre-gated on total B cells (CD19⁺CD3⁻). As shown in FIG. 20D, the frequency of naïve (CD38⁻CD27⁻), GC (CD38⁺CD27⁺), PB (CD38⁺⁺CD27⁺⁺), Memory (CD38⁻CD27⁺), and Pre-GC (CD38⁺CD27⁻) B cells in control, FOXP3 KO, and GzmB KO tonsil organoids were compared after 10-day culture. Representative data from four independent experiments. The mean±SEM is indicated. *p<0.05, **p<0.01 (Paired t-test).

FIGS. 21A-21B show FOXP3 KO and GZMB KO tonsil organoids developed differential autoimmune phenotypes after stimulation with LAIV and autoantigen stimulation. FIG. 21A shows the fold change of the O.D. value represents the amount of autoantibody specific to Proteinase 3 (PR3), double-stranded DNA (dsDNA), small nuclear ribonucleoprotein 70 kDa (snRNP70), and core histone from control, FOXOP3 KO, and GzmBKO tonsil organoids. Data from three independent experiments. The mean±SEM is indicated. *p<0.05, **p<0.01 (Paired t-test). FIG. 21B shows the percentage of PB cells (CD27⁺⁺CD38⁺⁺ B cells), activated CD4⁺ and CD8⁺ T cells (CD38⁺CD27⁺) from non-stimulated (NS), LAIV stimulation, LAIV plus autoantigens (LAIV+A) stimulation from control, FOXP3 KO, GZMB KO tonsil organoids after 10-day culture. Data from three independent experiments. The mean±SEM is indicated. *p<0.05 (Unpaired t-test).

FIG. 22 shows that when live attenuated flu vaccine (LAIV) was used as an adjuvant in the FoxP3 KO and Granzyme B (BrzB) KO, the resulting bulk anti influenza HA affinities were measured using interferometry. There was a marked increase in the affinity in most donors (n=9) with the FoxP3 KO, up to an 18-fold decrease in the off rate (k_{d}), with a more modest effect of the GrzB KO in a few donors.

### DETAILED DESCRIPTION

Disclosed herein are methods, systems, and devices capable of recapitulating an immune responses and function, e.g. production of high affinity antibodies.

The methods, systems and devices can comprise immune cell structures capable of, for example, modeling adaptive immune responses including antigen-specific hypermutation, affinity maturation, and class switching of B cells. The immune cell structures can comprise an in vitro cell cluster comprising lymphoid cells. The lymphoid cells can be derived from human tonsil tissue. The lymphoid cells can be derived from human spleen tissue. The in vitro cell cluster can be in contact with a cell culture media in a Transwell system, with the cells suspended in media on a porous membrane. The in vitro cell culture can be held at a temperature at or around ninety-eight degrees Fahrenheit. The in vitro cell cluster can be exposed to one or more adjuvants. The in vitro cell cluster can be exposed to one or more antigens. The antigen can be a protein, carbohydrate, glycoprotein, nucleic acid, mRNA encoding a protein, or fragment thereof. The antigen can be a viral protein, a growth factor, a cancer related protein, or an auto-immune disease related protein.

The in vitro cell cluster can comprise a spatial organization of lymphoid tissue. The spatial organization can comprise a germinal center. The spatial organization can comprise an aggregate of T-cells and a distribution of rare but essential non-lymphoid cells. The in vitro cell cluster can be configured to maintain a spatial organization and cellular respiration for at least 24 hours.

The in vitro cell cluster can be configured to perform one or more of: hypermutation maturation, affinity maturation, plasmablast differentiation, class switching recombination, and antigen-specific antibody production. The cells can be configured to differentiate to form said in vitro cell cluster upon exposure to an antigen. The germinal center can comprise antigen presenting cells (APCs) and T-cells at least partially surrounding said functional germinal center. The APCs can comprise B-cells or dendritic cells. The dendritic cells can comprise follicular dendritic cells. The B-cells can comprise CD38+ B-cells. The B-cells can comprise CD27+ B-cells. The T-cells can comprise CD8+ T-cells. The T-cells can comprise CD4+ T-cells.

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential methods and materials are now described.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The term “subject,” as used herein, generally refers to any animal or living organism. Animals can be mammals, such as humans, non-human primates, rodents such as mice and rats, dogs, cats, pigs, sheep, rabbits, and others. Animals can be fish, reptiles, or others. Animals can be neonatal, infant, adolescent, or adult animals. A human may be an infant, a toddler, a child, a young adult, an adult or a geriatric. A human can be more than about 1, 2, 5, 10, 20, 30, 40, 50, 60, 65, 70, 75, or about 80 years of age.

### Cell Culture Conditions

The in vitro cell cluster can be in contact with a media. The media can comprise one or more components. The first component can comprise a media component comprised of AIM V, IMDM, MEM, DMEM, RPMI 1640, Alpha Medium or McCoy's Medium, or an equivalent known culture medium component. The second can be a serum component which can comprise human serum, fetal bovine serum, horse serum, or serum-free replacement. The third component can be an activating factor which can comprise B-cell activating factor (BAFF). The fourth component can be an antibiotic to prevent microbial growth. The fifth component can be basal media supplements which can comprise nonessential amino acids, sodium pyruvate, insulin/selenium/transferrin cocktail, growth factors, hormones, or cytokines. The media can be supplemented with fetal calf serum. The media can be replaced every one day, two days, three days, or four days. The in vitro cell cluster can be in contact with a cell culture media in a Transwell system, with the cells suspended in media on a porous membrane.

### Antigens

The cells can be configured to differentiate to form said in vitro cell cluster upon exposure to an antigen. The antigen can be any substance that binds to an antibody. Antigens can be originated from the environment or formed inside the body. The antigen can be a peptide, protein or fragment thereof, polysaccharides, lipids, nucleic acids, or other biomolecules. The protein can be a viral protein, a bacterial protein, a growth factor, a cancer related protein, a cancer related peptide, an auto-immune disease related protein, an auto-immune disease related peptide, or fragment thereof. The antigen can be an auto-antigen.

The antigen can be a protein or fragment thereof. The protein can be a viral protein, a growth factor, a cancer related protein, a bacterial antigen, a fungal antigen, or an auto-immune disease related protein. The viral protein can be derived from a virus. The viral protein can be derived from, for example, a helical virus, a polyhedral virus, a spherical virus, or a complex virus. The antigen can be a virus. The virus can be, for example, a coronavirus or a flu virus, adenovirus, megavirus, Epstein-Barr virus, Adenovirus, Coxsackievirus, Megavirus, Nipah virus, Marburgvirus, Hepatitis C virus, Influenza A virus, Varicella zoster virus, Canine parvovirus, Hepatitis B virus, Rabies virus, Monkeypox virus, Human coronavirus HKU1, Dengue virus, Human immunodeficiency virus 1, Severe acute respiratory syndrome-related coronavirus, Middle East respiratory syndrome-related coronavirus, Feline immunodeficiency virus, Feline leukemia virus, Human metapneumovirus, Parvovirus B19, Human polyomavirus 2, Feline calicivirus, Eastern equine encephalitis virus, BK virus, Hendra virus, Norwalk virus, Ross River virus, Variola virus, Vaccinia virus, Herpes simplex virus 1, Kaposi's sarcoma-associated herpesvirus, Mumps virus, Measles morbillivirus, SV40, Human coronavirus 229E, Cyprinid herpesvirus 3, B virus, Betaarterivirus suid 1, Indiana vesiculovirus, Hepatitis A virus, Human herpesvirus 5, Bacteriophage MS2, Enterobacteria phage T2, Human coronavirus NL63, Murine respirovirus, Human herpesvirus 2, Simian immunodeficiency virus, or Equid alphaherpesvirus 1.

The antigen can be a vaccine or vaccine candidate. Vaccines known and used in the art include, but are not limited to, inactivated pathogen vaccines; live-attenuated pathogen vaccines; messenger RNA (mRNA) vaccines; subunit, recombinant, polysaccharide, and conjugate vaccines; toxoid vaccines; and viral vector vaccines. Inactivated vaccines use a killed version of the pathogen that causes a disease, e.g. Hepatitis A, influenza, rabies, etc. Live vaccines use an attenuated form of the pathogen that causes a disease, e.g. measles, mumps, rubella (MMR combined vaccine), rotavirus, smallpox, chickenpox, yellow fever. mRNA vaccines encode pathogen proteins that trigger an immune response, e.g. SRS-CoV2. Subunit, recombinant, polysaccharide, and conjugate vaccines use specific pathogen molecules, e.g. Hib (Haemophilus influenzae type b), Hepatitis B, HPV (Human papillomavirus), Bordetella pertussis, pneumococcal disease, meningococcal disease, Varicella Zoster virus. Toxoid vaccines use a toxin made by the pathogen, e.g. diphtheria, and tetanus. Viral vector vaccines use a modified version of a different virus as a vector to deliver sequences encoding pathogen protein. Several different viruses have been used as vectors, including influenza, vesicular stomatitis virus (VSV), measles virus, and adenovirus. Viral vectors are in use currently for SARS-COV2.

### Adjuvants

Immune responses of the immunological organoid model as described herein can be enhanced by the use of an adjuvant or derivative thereof. Adjuvants can comprise, for example, aluminum salts, Freund's adjuvant, Poly-IC, Poly-ICLC, MDP, MPL, CpG ODN, Virosome, MF59, AS01, Flagellin, R837/R848, AS04, AS02, AS03, mineral adjuvants such as aluminum hydroxide, phosphate adjuvants, calcium phosphate adjuvants, imiquimod, ISA51, or any combination thereof.

The lymphoid organoid can be incubated with an antigen and an adjuvant. The lymphoid organoid can be incubated with an antigen before introducing the adjuvant. The lymphoid organoid can be incubated with an antigen and/or adjuvant for at least about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 49 hours, 50 hours, 51 hours, 52 hours, 53 hours, 54 hours, 55 hours, 56 hours, 57 hours, 58 hours, 59 hours, 60 hours, 61 hours, 62 hours, 63 hours, 64 hours, 65 hours, 66 hours, 67 hours, 68 hours, 69 hours, 70 hours, 71 hours, 72 hours, or more. The incubating can be for at least 48 hours. Incubation with one or more antigens can increase the percent of antigen specific B-cells by at least 1%, 2%, 3%, 4%, 5%, 6%, 7%, 8%, 9%, 10%, 11%, 12%, 13%, 14%, 15%, 20%, 25%, 30%, 35%, 40%, 50%, 60%, 65%, 70%, 75%, 80%, 85%, 90%, 95%, 100% or more as compared to before incubation.

Incubation with one or more antigens can increase a number of nucleotide mutations from a germline heavy chain BCR sequence by at least 1%, 2%, 3%, 4%, 5%, 6%, 7%, 8%, 9%, 10%, 11%, 12%, 13%, 14%, 15%, 20%, 25%, 30%, 35%, 40%, 50%, 60%, 65%, 70%, 75%, 80%, 85%, 90%, 95%, 100% or more as compared to before incubation.

Incubation with one or more antigens can increase a percent of antibody secreting B-cells amongst the total population of B cells by at least 1%, 2%, 3%, 4%, 5%, 6%, 7%, 8%, 9%, 10%, 11%, 12%, 13%, 14%, 15%, 20%, 25%, 30%, 35%, 40%, 50%, 60%, 65%, 70%, 75%, 80%, 85%, 90%, 95%, 100% or more as compared to before incubation.

### Cell Types

The immunological organ model can comprise an in vitro cell cluster. The cell cluster can comprise lymphoid cells, e.g. B cells and T cells. Lymphoid cells can be derived from a lymphoid organ. The lymphoid cells can be derived from tonsil tissue, spleen tissue, adenoid tissue, thymus tissue, or lymph node tissue. Lymphoid organs can include, for example, primary and secondary lymphoid organs. Lymphoid organs can comprise bone marrow, thymus, lymph nodes, spleen, tonsils, tissues in mucous membrane layers of the body (e.g., bowel, respiratory and urinary tracts, and the lining of the vagina), small intestine (Peyer's patches), appendix. The in vitro-cell cluster can comprise a germinal center. The in vitro cell cluster can comprise a T-cell aggregate.

The method can further comprise obtaining said lymphoid cells from a subject. The lymphoid cells can be obtained by biopsy, swab, or aspiration. The lymphoid cells can be derived from tonsil tissue, spleen tissue, adenoid tissue, thymus tissue, or lymph node tissue. The lymphoid cells can be derived from a lymphoid organ. Lymphoid organs can include, for example, primary and secondary lymphoid organs. Lymphoid organs can comprise bone marrow, thymus, lymph nodes, spleen, tonsils, tissues in mucous membrane layers of the body (e.g., bowel).

The lymphoid cells, e.g. T-cells, can be modified to reduce or eliminate expression of a forkhead box transcription factor. The forkhead box transcription factor can be FoxP3. The lymphoid cells, e.g. T-cells, can be modified to reduce or eliminate expression of a granzyme B (GZMB). The lymphoid cells can be modified by in vitro programmed genome editing, e.g. with a CRISPR-based system.

Cells may be separated from a mixture of cells by techniques that enrich for desired cells, or may be engineered and cultured without separation. An appropriate solution may be used for dispersion or suspension. Such solution will generally be a balanced salt solution, e.g. normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc.

Techniques for affinity separation may include magnetic separation, using antibody-coated magnetic beads, affinity chromatography, cytotoxic agents joined to a monoclonal antibody or used in conjunction with a monoclonal antibody, e.g., complement and cytotoxic cells, and “panning” with antibody attached to a solid matrix, e.g., a plate, or other convenient technique. Techniques providing accurate separation include, but are not limited to, fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g., propidium iodide).

The collected and optionally enriched cell population may be used immediately for genetic modification, or may be frozen at liquid nitrogen temperatures and stored, being thawed and capable of being reused. The cells will usually be stored in 10% DMSO, 50% FCS, 40% RPMI 1640 medium.

A population of lymphoid cells can be genetically modified, e.g. to comprise genetically modified T cells. The genetically modified T-cells can be regulatory T-cells. The genetically modified T-cells can be modified to knock down or knock out expression of a forkhead box transcription factor. The forkhead box transcription factor can be FoxP3. The genetically modified T-cells can be modified to knock down or knock out expression of granzyme B (GXMB). The genetically modified T-cells can be CD8⁺ T-cells.

Gene editing, or genome editing, is a type of genetic engineering in which DNA is inserted, replaced, or removed from a genome using nucleases. The nucleases may be artificially engineered. Alternately, the nucleases may be found in nature. The nucleases create specific double-stranded breaks (DSBs) at desired locations in the genome. The cell's endogenous repair mechanisms subsequently repair the induced break(s) by natural processes, such as homologous recombination (HR) and non-homologous end-joining (NHEJ). Nucleases include, but are not limited to, Zinc Finger Nucleases (ZFNs), Transcription Activator-Like Effector Nucleases (TALENs), CRISPR, (e.g., the CRISPR/Cas system), and engineered meganuclease re-engineered homing endonucleases. CRISPR nucleases include, but are not limited to, a Cas nuclease, a Cpf1 nuclease, a C2c1 nuclease, a C2c3 nuclease, and a C2c3 nuclease.

In an embodiment, the nuclease comprises a CRISPR/Cas system. The CRISPR (clustered regularly interspaced short palindromic repeats) locus, which encodes RNA components of the system, and the Cas (CRISPR-associated) locus, which encodes proteins (Jansen et al., 2002. Mol. Microbiol. 43: 1565-1575; Makarova et al., 2002. Nucleic Acids Res. 30:482-496; Makarova et al., 2006. Biol. Direct 1: 7; Haft et al., 2005. PLoS Comput. Biol. 1: e60) make up the gene sequences of the CRISPR/Cas nuclease system. CRISPR loci in microbial hosts contain a combination of CRISPR-associated (Cas) genes as well as non-coding RNA elements capable of programming the specificity of the CRISPR-mediated nucleic acid cleavage.

The Type II CRISPR is one of the most well characterized systems and carries out targeted DNA double-strand breaks in four sequential steps. First, two non-coding RNAs, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA: tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer. Activity of the CRISPR/Cas system comprises of three steps: (i) insertion of alien DNA sequences into the CRISPR array to prevent future attacks, in a process called “adaptation”, (ii) expression of the relevant proteins, as well as expression and processing of the array, followed by (iii) RNA-mediated interference with the alien nucleic acid.

In certain embodiments, Cas protein may be a “functional derivative” of a naturally occurring Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof. Suitable derivatives of a Cas polypeptide or a fragment thereof include but are not limited to mutants, fusions, covalent modifications of Cas protein or a fragment thereof. Cas protein, which includes, but is not limited to, Cas protein or a fragment thereof, as well as derivatives of Cas protein or a fragment thereof, may be obtainable from a cell or produced in vitro or by a combination of these two procedures. The cell may be a cell that naturally produces Cas protein or a cell that naturally produces Cas protein and is genetically engineered to produce the endogenous Cas protein at a higher expression level or to produce a Cas protein from an exogenously introduced nucleic acid, which encodes a Cas that is the same as or different from the endogenous Cas. In some cases, the cell does not naturally produce Cas protein and is genetically engineered to produce a Cas protein.

The method also includes introducing single-guide RNAs (sgRNAs) into the lymphoid cell population. The guide RNAs (sgRNAs) include nucleotide sequences that are complementary to the target chromosomal DNA. The sgRNAs can be, for example, engineered single chain guide RNAs that comprise a crRNA sequence (complementary to the target DNA sequence) and a common tracrRNA sequence, or as crRNA-tracrRNA hybrids. The sgRNAs can be introduced into the cell or the organism as a DNA (with an appropriate promoter), as an in vitro transcribed RNA, or as a synthesized RNA. The target DNA sequence can be a FoxP3 sequence. The target sequence can be a granzyme B sequence.

### Germinal Center Features

The in vitro cell cluster can be configured to perform one or more of: somatic hypermutation, affinity maturation, plasmablast differentiation, class switching recombination, and antigen-specific antibody production. In order to develop antigen specificity, in the germinal center, B cells can undergo a process called somatic hypermutation where point mutations are introduced into B cell receptor (BCR) gene sequence of the antibody variable regions of both the heavy and light chains at a very high rate compared to the background mutation rates observed in other genes. These mutated B cells are different from each other in specificity of antigen. To ensure high affinity of antibody production, mutated B cell are further selected based on the binding affinity of their receptors to antigen from follicular dendritic cells (FDCs), macrophages, or dendritic cells. Those B cells that have a negative effect on antigen binding undergo apoptosis while those with positive binding affinity are selected. This process is called affinity maturation. These two events result in a generation of B cells whose BCRs bind to specific antigen with high affinity. Selected B cells then differentiate into memory B cells or plasmablasts, which are also known as antibody secreting cells (ASC). Plasmablasts produce a large amount of antibodies during the first wave before undergoing apoptosis within a few days while memory B cells provide longer immunity. Memory B cells can also secrete different class of antibodies or immunoglobins (Ig) via a process called class switching recombination. During this process, a DNA recombination process of the constant region of the antibody heavy chain is changed while the variable region of the heavy chains remains the same. As a result, the antibody retains affinity for the same antigen but can interact with different effector molecules.

The germinal center can comprise antigen presenting cells (APCs), for example “professional” antigen presenting cells. The germinal center can be at least partially surrounded by T-cells. The APCs can comprise B-cells, macrophages, or dendritic cells. The dendritic cells can comprise follicular dendritic cells, pDC. The B-cells can comprise, for example, CD3− B-cells, CD45+ B-cells, CD19+ B-cells, CD38+ B-cells, CD38− B-cells, or CD27+ B-cells. The B-cells can comprise transitional B-cells, naïve B-cells, plasma B-cells, memory B-cells, pre-GC B cells, GC B cells, plasmablasts. The T-cells can comprise, for example, helper T-cells, follicular helper T-cells, follicular regulatory T-cells, cytotoxic T-cells, memory T-cells, regulator T-cells, natural killer T-cells, mucosal associated invariant T-cells, gamma delta T-cells. The T-cells can comprise CD8+ T-cells, CD4+ T-cells.

The germinal center can comprise a dark zone and a light zone. The dark zone can contain about 5%, 10%, 15%, 30%, 40%, 50%, 60%, 70%, 80%, 90%, 100%, or more CXCR4+ B cells compared to the light zone. The light zone can contain about 5%, 10%, 15%, 30%, 40%, 50%, 60%, 70%, 80%, 90%, 100%, or more CD83+ B cells compared to the dark zone. At least about 1%, 2%, 3%, 4%, 5%, 10%, 15%, 30%, 40%, or more cells in the dark zone can be CXCR4+ B cells. At least about 1%, 2%, 3%, 4%, 5%, 10%, 15%, 30%, 40%, or more cells in the light zone can be CD83+ B cells. The germinal center can comprise at least about 1%, 2%, 3%, 4%, 5%, 10%, 15%, 30%, or more CD83+ B-cells. The germinal center can comprise at least about 1%, 2%, 3%, 4%, 5%, 10%, 15%, 30%, or more CXCR4+ B-cells. The light zone can contain about 5%, 10%, 15%, 30%, 40%, 50%, 60%, 70%, 80%, 90%, 100%, or more CD86+ cells. CXCL12-expressing reticular cells (CRCs) are stromal cells known to reside in the dark zone. The dark zone can contain about 5%, 10%, 15%, 30%, 40%, 50%, 60%, 70%, 80%, 90%, 100%, or more CRCs.

### Antibodies

The term “antibody” herein is used in the broad sense and specifically covers monoclonal antibodies, polyclonal antibodies, monomers, dimers, multimers, multispecific antibodies (e.g., bispecific antibodies), heavy chain only antibodies, three chain antibodies, single chain Fv, nanobodies, etc., and also include antibody fragments, so long as they exhibit the desired biological activity (Miller et al (2003) Jour. of Immunology 170:4854-4861). Antibodies may be murine, human, humanized, chimeric, or derived from other species, usually human antibodies are produced by the cultures described herein

The term antibody may reference a full-length heavy chain, a full length light chain, an intact immunoglobulin molecule; or an immunologically active portion of any of these polypeptides, i.e., a polypeptide that comprises an antigen binding site that immunospecifically binds an antigen of a target of interest or part thereof, such targets including but not limited to, cancer cell or cells that produce autoimmune antibodies associated with an autoimmune disease. An antibody can be of any type (e.g., IgG, IgE, IgM, IgD, and IgA), class (e.g., IgG1, IgG2, IgG3, IgG4, IgA1 and IgA2) or subclass of immunoglobulin molecule. The immunoglobulins can be derived from any species. In one aspect, the immunoglobulin is of human origin.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat et al (1991) Sequences of Proteins of Immunological Interest, 5th Ed. Public Health Service, National Institutes of Health, Bethesda, Md.). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region may comprise amino acid residues from a “complementarity determining region” or “CDR”, and/or those residues from a “hypervariable loop”. “Framework Region” or “FR” residues are those variable domain residues other than the hypervariable region residues as herein defined.

Variable regions of interest include 3 CDR sequences, which may be obtained from available antibodies with the desired specificity, or may be obtained from antibodies developed for this purpose. One of skill in the art will understand that a number of definitions of the CDRs are commonly in use, including, but not limited to, the Kabat definition (see “Zhao et al. A germline knowledge based computational approach for determining antibody complementarity determining regions.” Mol Immunol. 2010; 47:694-700), which is based on sequence variability and is the most commonly used. The Chothia definition is based on the location of the structural loop regions (Chothia et al. “Conformations of immunoglobulin hypervariable regions.” Nature. 1989; 342:877-883). Alternative CDR definitions of interest include, without limitation, those disclosed by Honegger, “Yet another numbering scheme for immunoglobulin variable domains: an automatic modeling and analysis tool.” J Mol Biol. 2001; 309:657-670; Ofran et al. “Automated identification of complementarity determining regions (CDRs) reveals peculiar characteristics of CDRs and B cell epitopes.” J Immunol. 2008; 181:6230-6235; Almagro “Identification of differences in the specificity-determining residues of antibodies that recognize antigens of different size: implications for the rational design of antibody repertoires.” J Mol Recognit. 2004; 17:132-143; and Padlanet al. “Identification of specificity-determining residues in antibodies.” Faseb J. 1995; 9:133-139, each of which is herein specifically incorporated by reference.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations, which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method.

An “intact antibody chain” as used herein is one comprising a full length variable region and a full length constant region. An intact “conventional” antibody comprises an intact light chain and an intact heavy chain, as well as a light chain constant domain (CL) and heavy chain constant domains, CH1, hinge, CH2 and CH3 for secreted IgG. Other isotypes, such as IgM or IgA may have different CH domains. The constant domains may be native sequence constant domains (e.g., human native sequence constant domains) or amino acid sequence variants thereof. The intact antibody may have one or more “effector functions” which refer to those biological activities attributable to the Fc constant region (a native sequence Fc region or amino acid sequence variant Fc region) of an antibody. Examples of antibody effector functions include, but are not limited to, C1q binding; complement dependent cytotoxicity; Fc receptor binding; antibody-dependent cell-mediated cytotoxicity (ADCC); phagocytosis (ADCP); and down regulation of cell surface receptors. Constant region variants include those that alter the effector profile, binding to Fc receptors, and the like.

Depending on the amino acid sequence of the constant domain of their heavy chains, intact antibodies can be assigned to different “classes.” There are five major classes of intact immunoglobulin antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known. Ig forms include hinge-modifications or hingeless forms (Roux et al (1998) J. Immunol. 161:4083-4090; Lund et al (2000) Eur. J. Biochem. 267:7246-7256; US 2005/0048572; US 2004/0229310). The light chains of antibodies from any vertebrate species can be assigned to one of two clearly distinct types, called κ and λ, based on the amino acid sequences of their constant domains.

“Affinity” refers to the strength of binding, increased binding affinity being correlated with a lower KD. In an embodiment, affinity is determined by surface plasmon resonance (SPR), e.g. as used by Biacore systems. The affinity of one molecule for another molecule can be determined by measuring the binding kinetics of the interaction, e.g. at 25° C. A “high affinity” antibody may bind to its cognate antigen with a K_{D} of 0.1 μM or better, a K_{D} of 0.01 μM or better, a K_{D} of 1 nM or better, a K_{D} of 0.1 nM or better, a K_{D} of 0.01 nM or better, a K_{D} of 1 pM or better, a K_{D} of 0.1 pM or better, a K_{D} of 0.01 pM or better.

### Cell Viability

The spatial organization of lymphoid tissue can be maintained for at least about twelve hours, one day, two days, three days, four days, five days, six days, seven days, eight days, nine days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or more. The cell cluster can maintain a cellular respiration for at least about twelve hours, one day, two days, three days, four days, five days, six days, seven days, eight days, nine days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or more. Cellular respiration can be measured by, for example, a resazurin reduction assay, protease viability marker assay, ATP assay, or luciferase assay.

### Self-Antigens and Autoantibodies

Autoimmune disease is a condition in which the immune cells mistakenly attack healthy cells in the body. Some autoimmune diseases are caused by autoantibodies (or natural antibodies), which are antibodies produced against substances (e.g., expressed proteins) formed by an individual's own body. Self-antigens (or self-antigens) are substances (e.g., expressed proteins) that stimulate autoantibodies. These substances can be found in all cell types or be highly specific to a certain cell type in one tissue. Self-antigens can comprise proteins, nucleic acids, carbohydrates, lipids, and various combination thereof. Most natural autoantibodies are polyreactive, meaning that they bind to several unrelated antigens with moderate affinity, typically in the low nanomolar range. However, through somatic hypermutation and class switching, autoantibodies can be expressed, and antibody affinity can be increased (e.g. in the femtomolar rage). Self-antigens can comprise, for example, Pr3, dsDNA, core histone, or SNRNP70.

Auto-antibodies disclosed herein can have an affinity to an auto-antigen greater than 1 nanomolar, 0.1 nanomolar, 0.01 nanomolar, 0.001 nanomolar, 0.0001 nanomolar, 1000 femtomolar, 100 femtomolar, 10 femtomolar, or 1 femtomolar. Auto-antibodies disclosed herein can have an affinity to an auto-antigen between 1 nanomolar and 10000 femtomolar, between 100000 femtomolar and 100 femtomolar, between 1000 femtomolar and 10 femtomolar, or between 100 femtomolar and 1 femtomolar.

### Transcription Factor Expression

In some embodiments, organoid cells are genetically modified to knock down or knock out expression of a transcription factor. In some embodiments, regulatory T-cells are genetically modified to knock down or knock out expression of a transcription factor. In some embodiments, T-cells are genetically modified to not regulate the production of antibodies by B-cells. In some embodiments, T-cells are genetically modified to allow the production of auto-antibodies by B-cells. In some embodiments, T-cells are genetically modified to allow the production of high affinity antibodies by B-cells. In some embodiments, the transcription factors are in the forkhead-box family of transcription factors. The Forkhead-box family of transcription factors play a role in regulating the expression of genes involved in cell growth, proliferation, differentiation, and longevity. Forkhead Box P3 (FOXP3) gene encodes FOXP3 transcriptional regulator, which is important for the development and inhibitory function of regulatory T-cells. FOXP3 is required for effective maintenance of tolerance and prevention of autoimmune diseases throughout the body. In some embodiments, T-cells are modified to reduce or inactivate an expression of FOXP3. FOXP3 gene inactivation can comprise knockout (KO) method or knockdown method and can be permanently or transiently. In some embodiments, FOXP3-knock out or knock down T-cells can be edited by any gene expression modification system known to one of skill in the art.

### Granzyme B Expression

In some embodiments, organoid cells are genetically modified to knock down or knock out expression of a cytolytic protein. In some embodiments the cells are T-cells. In some embodiments the cytolytic protein is Granzyme B. In some embodiments, T-cells are CD8⁺ T-cells. In some embodiments, T-cells are CD4⁺ T-cells. Granzyme B (GZMB) is a serine protease known for its perforin-dependent pro-apoptotic function underlying the capability of cytotoxic immune cells, as cytotoxic T lymphocytes (CTLs) and natural killer (NK) cells. Granzyme B exerts a perforin-dependent intracellular activity, and an extracellular perforin-independent function, consisting in the cleavage of multiple extracellular substrates, as extracellular matrix (ECM) components, cytokines, cell receptors, angiogenic and clotting proteins.

### Gene Editing Systems

Methods of gene expression knock out or knock down can comprise any gene editing system known in the art. Gene editing systems can comprise, for example, zinc finger nucleases, transcription activator-like effector nuclease (TALEN), clustered regularly interspaced short palindromic repeats (CRISPR)/Cas nuclease, meganuclease, or prime editing. Gene editing systems can comprise, for example, siRNA, shRNA, DNA-based RNAi, antisense oligonucleotides, or CRISPR-mediated gene knockdown, CRISPR interference (CRISPRi) dCas9 without additional proteins, CRISPRi dCas9 in combination with other proteins, or Cas13 family enzymes. Gene editing systems can comprise, for example, microinjection, electroporation, lipofection, ultrasound, gene gun, viral delivery, hydrodynamic applications.

### Uses

Disclosed herein is a method for generating antibodies, for example high affinity antibodies, from a lymphoid cell culture, for example, lymphoid organoid comprising placing lymphoid cells in a media to produce said cell culture. The cell culture can comprise a particular spatial organization of lymphoid tissue. The cell culture can include, but is not limited to, one or more germinal centers. The lymphoid cell culture can comprise an aggregate of T-cells. The method can comprise introducing an antigen to the media to, for example, enhance the immune response of the cell culture to an antigen. The method can comprise incubating the cell culture with an antigen to generate antibodies. Antibodies generated from the cell culture can be isolated from said cell culture.

Genetic sequences encoding an antibody of interest, e.g. a high affinity antibody, an antibody specific for an auto-antigen, etc., can be identified by any convenient method sequenced; inserted into an expression vector; etc. for analysis of responsiveness, structure function analysis, production of a selected antibody, etc.

The coding sequences may be inserted into a vector for expression and/or integration. Many such vectors are available. The vector components generally include, but are not limited to, one or more of the following: an origin of replication, one or more marker genes, an enhancer element, a promoter, and a transcription termination sequence. Vectors include, but are not limited to, viral vectors, plasmid vectors, integrating vectors, and the like. Expression vectors can comprise a promoter that is recognized by the host organism and is operably linked to the antibody coding sequence. Transcription by higher eukaryotes may be increased by inserting an enhancer sequence into the vector. Enhancers are cis-acting elements of DNA, usually about from 10 to 300 bp in length, which act on a promoter to increase its transcription. Expression vectors for use in eukaryotic host cells will also contain sequences necessary for the termination of transcription and for stabilizing the mRNA. Such sequences are commonly available from the 5′ and, occasionally 3′, untranslated regions of eukaryotic or viral DNAs or cDNAs. Construction of suitable vectors containing one or more of the above-listed components employs standard techniques. Suitable host cells for cloning a construct for expressing the selected antibody include prokaryotic, yeast, or other eukaryotic cells described above. The expressed antibody can be isolated from the host cell, and purified as known in the art.

An antibody produced by the methods disclosed herein can be formulated with an a pharmaceutically acceptable carrier (one or more organic or inorganic ingredients, natural or synthetic, with which a subject agent is combined to facilitate its application). A suitable carrier includes, but is not limited to, sterile saline although other aqueous and non-aqueous isotonic sterile solutions and sterile suspensions known to be pharmaceutically acceptable are known to those of ordinary skill in the art.

An antibody can be formulated as a composition comprising a pharmaceutically acceptable excipient. The form depends on the intended mode of administration and application. The compositions can also include, but are not limited to, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include, but is not limited to, other carriers, adjuvants, or nontoxic, nontherapeutic, nonimmunogenic stabilizers and the like.

Pharmaceutically acceptable excipients, such as vehicles, adjuvants, carriers or diluents, are commercially available. Moreover, pharmaceutically acceptable auxiliary substances, such as pH adjusting and buffering agents, tonicity adjusting agents, stabilizers, wetting agents and the like, are commercially available. Any compound useful in the methods and compositions of the invention can be provided as a pharmaceutically acceptable base addition salt. “Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particularly organic bases are isopropylamine, diethylamine, ethanolamine, trimethylamine, dicyclohexylamine, choline and caffeine.

The methods and systems disclosed herein may be useful for generating monoclonal antibodies for testing and treatment of a variety of diseases, including cancer, autoimmune disease, and/or infectious processes, including viral infection, bacterial infection, microbial infection, or a combination thereof. The cancer disease can be, for example, chronic lymphocytic leukemia, Hodgkin's lymphoma, Non-Hodgkin's lymphoma, bowel cancer, head cancer, neck cancer, breast cancer, stomach cancer, melanoma, glioblastoma, colorectal cancer, lung cancer, kidney cancer, or ovarian cancer. The autoimmune disease can be rheumatoid arthritis, Crohn's disease, celiac disease, pernicious anemia, autoimmune vasculitis, myasthenia gravis, Sjogren's syndrome, Graves' disease, Addison's disease, inflammatory bowel disease, systemic lupus erythematosus, Type 1 diabetes, Lupus, Multiple sclerosis, or psoriasis.

In certain aspects, the methods and systems disclosed herein may be useful for determining a treatment course for a subject. For example, such methods and systems may involve screening a patient for an immune response to a treatment. The subject can be healthy. The patient can be positive for a viral infection. The subject can be positive for an auto-immune disease. The subject can be positive for a fungal infection. The subject can be positive for a bacterial infection. The methods and systems may involve a screen that can be used prophylactically to identify a vaccination for a patient. The methods and systems may involve a screen that can be used to identify an immune response within a particular population of individuals.

In certain aspects, the methods disclosed herein are useful for screening vaccine candidates. A method can comprise contacting a lymphoid organoid with an effective dose of a candidate vaccine comprising administering the candidate vaccine, which may comprise an adjuvant; and determining the antibody response produced by the lymphoid organoid. An antibody response can be measured by bulk specificity and affinity measurements. An antibody response can be measured by sequencing antibody coding mRNA in cells of the lymphoid organoid. An antibody response can be measured by isolating antibody clones and determining the specificity and affinity. The response elicited by a vaccine candidate can be compared to the response elicited by control antigens. The response elicited by a vaccine candidate can be compared to the response elicited by one or more different vaccine candidates. In some embodiments, a candidate vaccine or adjuvant is selected for development based on the ability to provide a specific and/or high affinity antibody response.

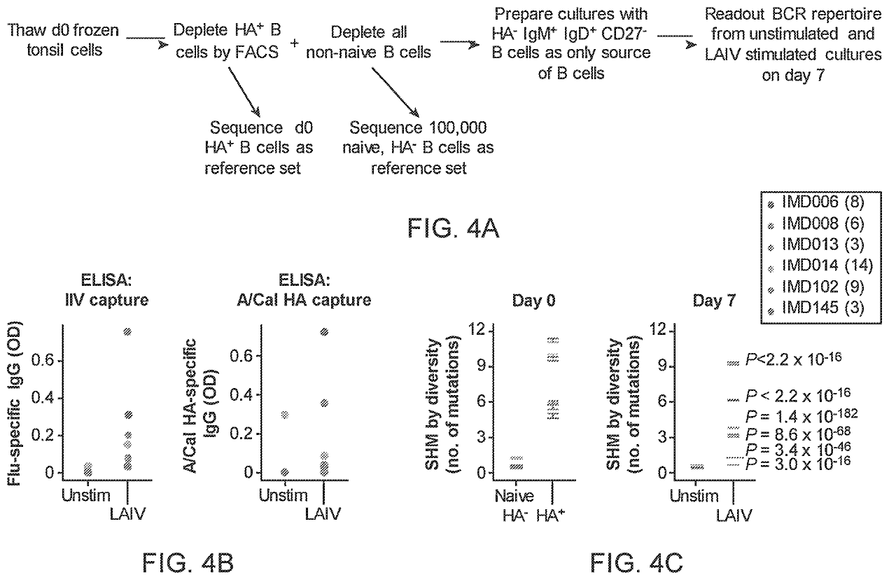

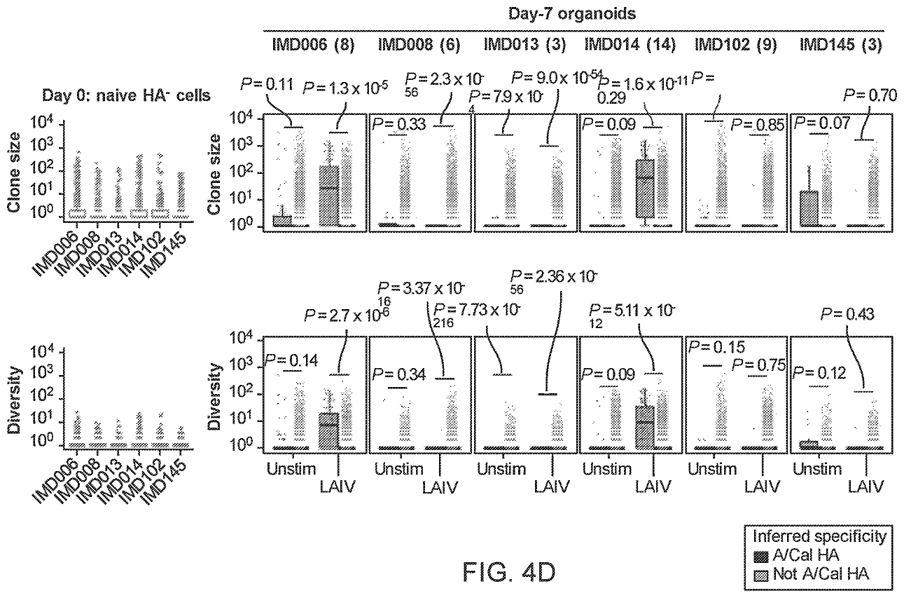

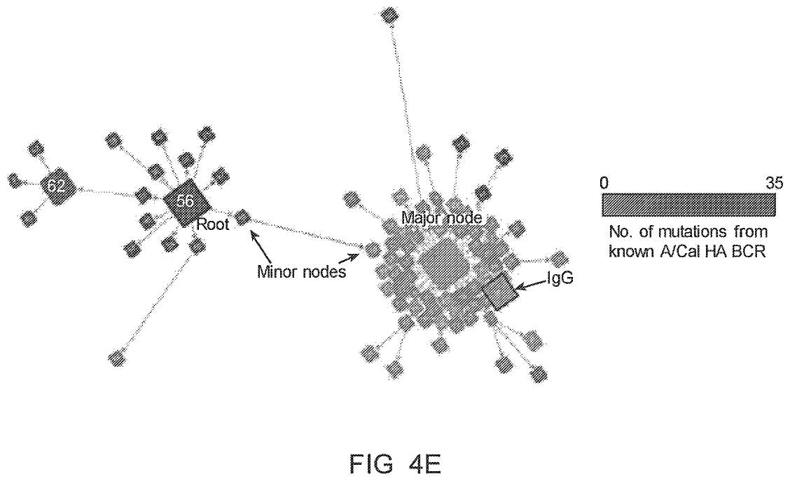

The present disclosure comprises an in vitro system supporting one or more of antigen-specific somatic hypermutation, affinity maturation and class switching of human B cells. Tonsils are a readily available and underutilized source of human lymphoid tissue and contain the cell types involved in adaptive immunity, including those largely absent from peripheral blood. Tonsil organoids were used to characterize features of the human influenza response and extended findings from previous human and murine studies. Preexisting HA-specific B cells had 5-10 heavy-chain nucleotide mutations (2-4% mutation rate), and LAIV-stimulated organoids prepared with naive-only B cells had 3-9 mutations, which was adequate to make high affinity specific antibodies (FIG. 4). These rates are in line with the average 5% mutation rate measured in influenza-vaccinated adults.

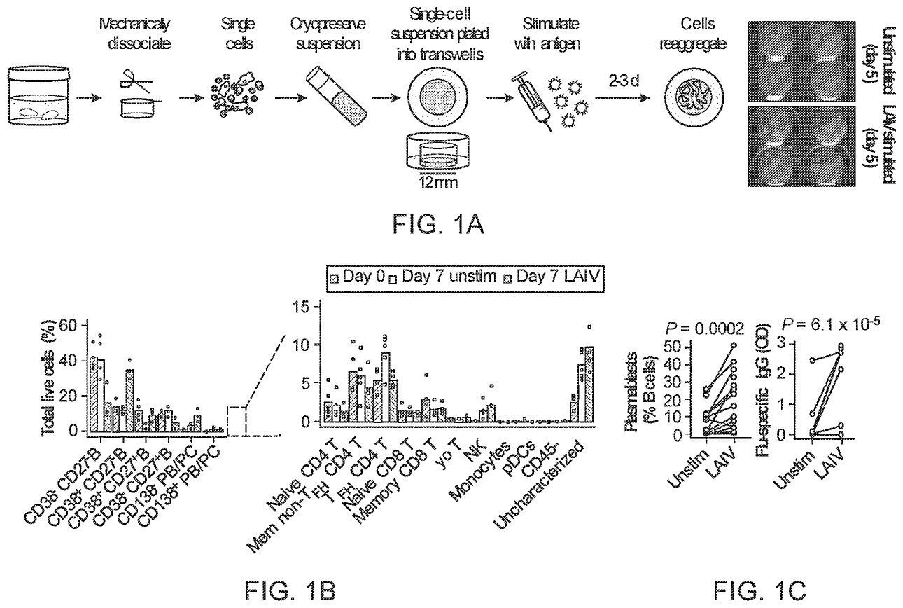

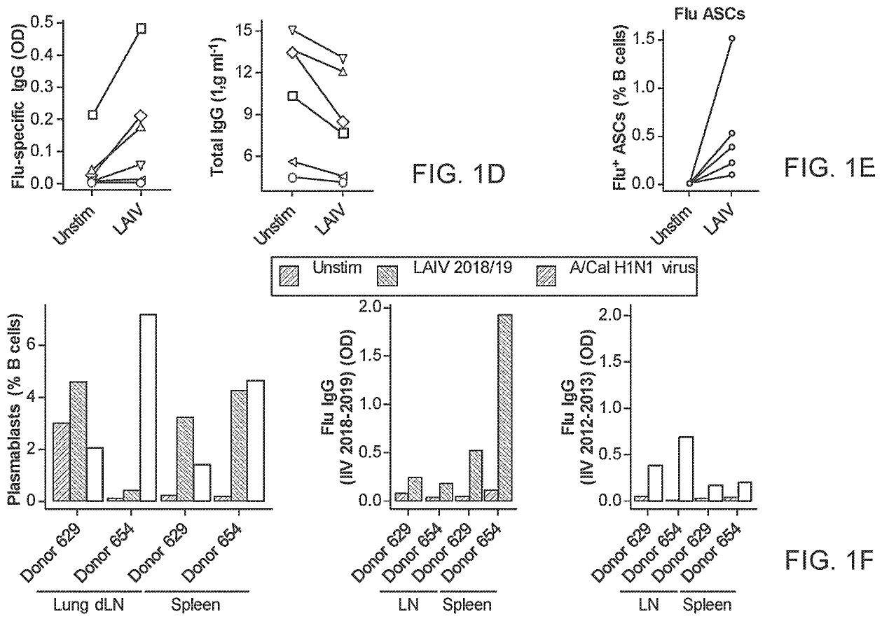

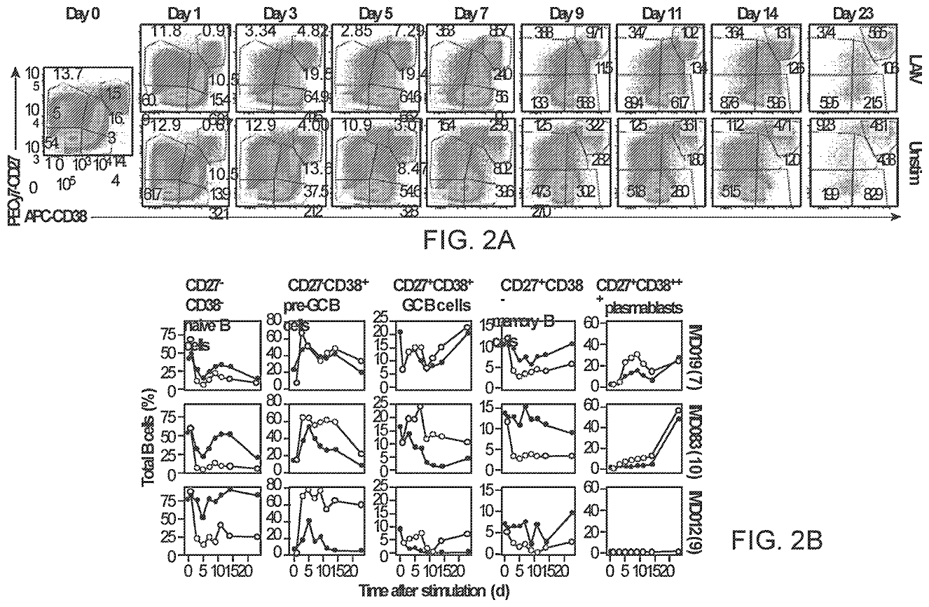

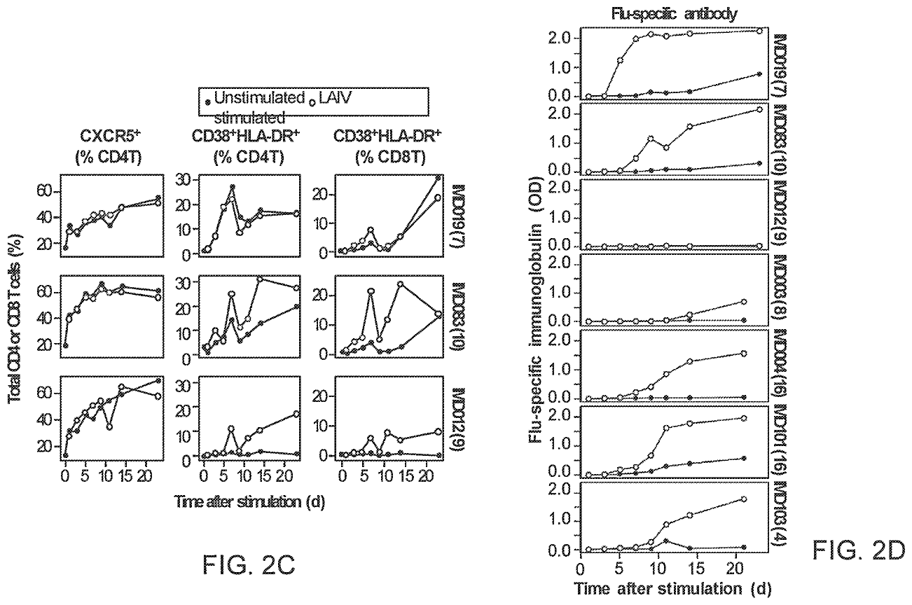

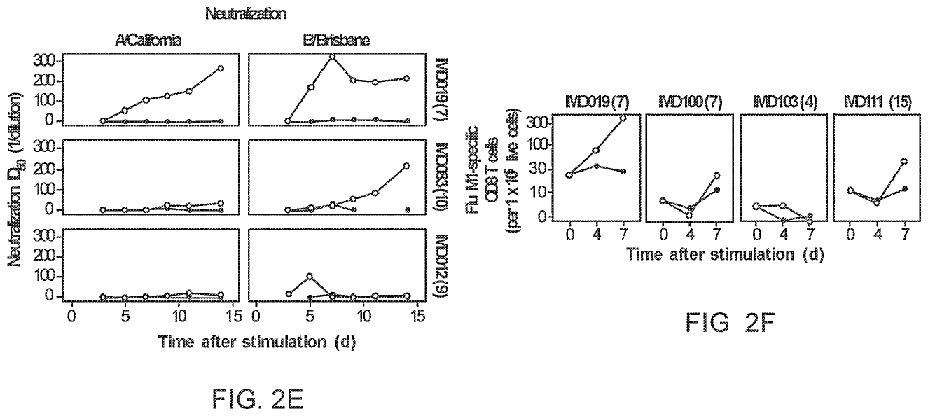

Human immune responses are incredibly variable, as seen in the current SARS-COV-2 pandemic, and a major goal of vaccine development is to confer protective immunity as broadly as possible. Another useful feature of the organoid system is its ability to assess immune response variability. While the organoids from most donors respond to LAIV, the magnitude and kinetics of those responses vary widely. In a test of 15 donors, organoids prepared from only one donor did not respond to LAIV within 7 d (FIGS. 1A-1F). This individual was young and not vaccinated against influenza, suggesting that they could be naive to influenza, and organoids prepared from their cells might require more time to mount a response. In the longitudinal analyses (FIGS. 2A-2F), a non-responder was identified who was treated with daily inhaled corticosteroids, demonstrating the sensitivity of the system to immunosuppressive agents. Using SARS-COV-2 vaccine candidates, even more variability was found, with some individuals unable to make B cell and/or T cell responses.

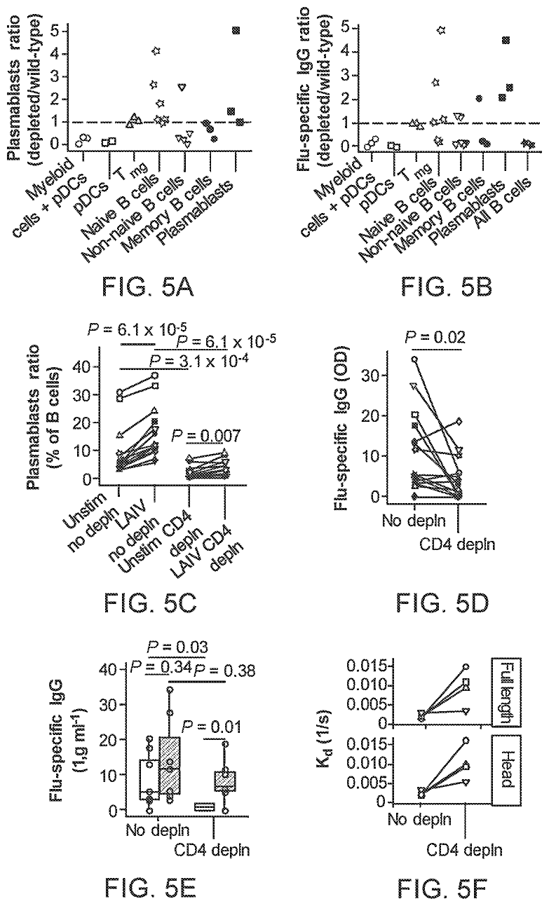

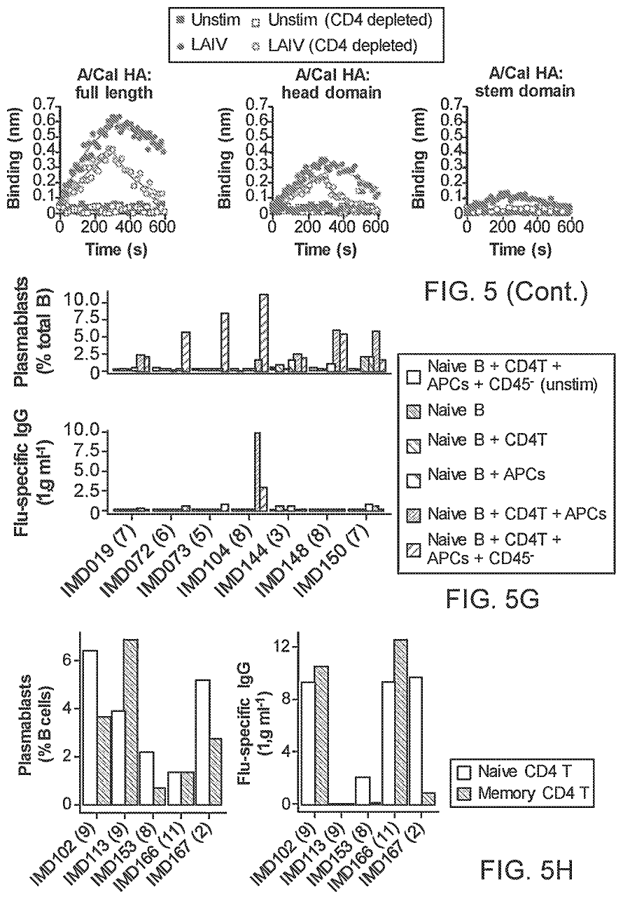

Using depletion studies, the functional relevance of individual immune cell types to the influenza response was established. pDCs were crucial to the antibody response, although they could be replaced by exogenous type I IFN. These data are consistent with a previous study that found that pDCs induce human blood B cell differentiation through type I IFN41 but contrast with a murine influenza challenge study that showed pDCs were dispensable to the response, highlighting a difference between murine and human systems. A role for preexisting plasmablasts/plasma cells in regulating B cell differentiation (FIGS. 5A-5B) was identified. When plasmablasts were depleted at the initiation of the culture, the influenza-specific antibody production was increased compared to wild-type controls. Secondary lymphoid tissues are competitive niches for plasma cell survival outside of the bone marrow, so depletion of preexisting plasma cells may promote antigen-specific plasmablast survival. It was demonstrated that T cell help plays an age-dependent role in the magnitude of the influenza-specific antibody response and showed that CD4+ T cells play an additional role in affinity maturation and B cell selection (FIGS. 5A-5H). Memory CD4+ T cells from older children better sustained a naive B cell response, suggesting that T cell help is refined by in vivo exposure. These findings are in line with an earlier mouse study showing that memory T cells can contribute to GC responses even though naive B cells typically did not in their system. Stem-specific antibodies were also only detectable when CD4+ T cells were present, suggesting that manipulating T cell help is a reasonable strategy to consider for future vaccine design.

Previous efforts to create artificial human lymphoid tissues depended on specialized bioreactors or complex in vitro differentiation strategies. Blood-derived mononuclear cells combined with monocyte-derived dendritic cells can stimulate IgM and cytokine secretion consistent with an adaptive immune response. It is questionable whether in vitro matured dendritic cells recapitulate the conditions that coordinate T and B cell responses in lymphoid tissues. More importantly, a major limitation of bioreactor systems is that they do not show mature GCs, class switching or affinity maturation, likely because many cells required for GC function are absent from peripheral blood mononuclear cells. Recent work illustrated that key aspects of the immune microenvironment can be captured in organoids in several studies that focused on conferring immune protection upon transplantation into mice or recapitulating features of the GC response. In these studies, designer hydrogel scaffolds for B cells combined with engineered fibroblasts were used to reveal transcriptional feedback loops involved in GC formation but lacked antigen specificity. Most recently, maleimide-functionalized hydrogel was shown to support GC B cell activation and an antigen-specific response in murine B cell organoid cultures. However, these methods do not incorporate autologous APCs nor T cells that are both crucial to refine the adaptive immune response in vivo. Due to the need for bioengineered fibroblast cell lines to provide necessary survival signals, these techniques are challenging to translate to human cells because of the diversity of human leukocyte antigen genes and generation of mixed leukocyte reactions. Previous techniques to produce in vitro models of human adaptive immunity have not been widely adopted because of reliance on specialized equipment or challenging technical protocols, poor throughput, lack of evidence for antigen-specific responses, affinity maturation and/or absence of cells known to be crucial to important features of adaptive immunity. Although humanized mice are increasingly used, they are expensive to procure and still have many limitations in terms of recapitulating human immunity.

It has been established that the LAIV is less effective than the inactivated formulation in adults, presumably due to the presence of preexisting antibodies at mucosal sites in non-naive individuals. Such conditions can be mimicked by introducing autologous serum or spiking in influenza-specific antibodies at the initiation of organoid culture.

### Kits

A kit may include, but is not limited to, one or more containers housing one or more of the components provided in this disclosure and instructions for use. Specifically, such kits may include, but is not limited to, one or more compositions described herein, along with instructions describing the intended application and the proper use and/or disposition of these compositions. Kits may comprise the components in appropriate concentrations or quantities for running various experiments.

### Machine Learning

The methods and systems disclosed herein can utilize artificial intelligence/machine learning to generate optimal antibodies with high specificity and affinity. Artificial intelligence/machine learning can be used to predict an antigen that might be specific to certain type of cancer, autoimmune disease or infection. The predicted antigen can be used in antibody production from the methods and systems disclosed in this invention.

### Computer Systems

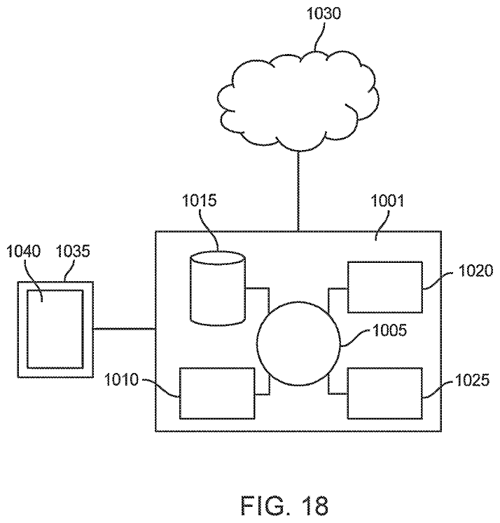

The present disclosure provides computer systems for implementing methods provided herein. FIG. 18 shows an example of a computer system 1001. The computer system 1001 includes, but is not limited to, a central processing unit (CPU, also “processor” and “computer processor” herein) 1005, which can be a single core or multi core processor, or a plurality of processors for parallel processing. The computer system 1001 also includes, but is not limited to, memory or memory location 1010 (e.g., random-access memory, read-only memory, flash memory), electronic storage unit 1015 (e.g., hard disk), communication interface 1020 (e.g., network adapter) for communicating with one or more other systems, and peripheral devices 1025, such as cache, other memory, data storage and/or electronic display adapters. The memory 1010, storage unit 1015, interface 1020 and peripheral devices 1025 are in communication with the CPU 05 through a communication bus (solid lines), such as a motherboard. The storage unit 1015 can be a data storage unit (or data repository) for storing data. The computer system 1001 can be operatively coupled to a computer network (“network”) 1030 with the aid of the communication interface 1020. The network 1030 can be the Internet, an internet and/or extranet, or an intranet and/or extranet that is in communication with the Internet. The network 1030 in some cases is a telecommunication and/or data network. The network 1030 can include, but is not limited to, one or more computer servers, which can enable distributed computing, such as cloud computing. The network 1030, in some cases with the aid of the computer system 1001, can implement a peer-to-peer network, which may enable devices coupled to the computer system 1001 to behave as a client or a server.

The CPU 1005 can execute a sequence of machine-readable instructions, which can be embodied in a program or software. The instructions may be stored in a memory location, such as the memory 1010. The instructions can be directed to the CPU 1005, which can subsequently program or otherwise configure the CPU 1005 to implement methods of the present disclosure. Examples of operations performed by the CPU 1005 can include fetch, decode, execute, and writeback.

The CPU 1005 can be part of a circuit, such as an integrated circuit. One or more other components of the system 1001 can be included in the circuit. In some cases, the circuit is an application specific integrated circuit (ASIC).

The storage unit 1015 can store files, such as drivers, libraries and saved programs. The storage unit 1015 can store user data, e.g., user preferences and user programs. The computer system 1001 in some cases can include one or more additional data storage units that are external to the computer system 1001, such as located on a remote server that is in communication with the computer system 1001 through an intranet or the Internet.

The computer system 1001 can communicate with one or more remote computer systems through the network 1030. For instance, the computer system 1001 can communicate with a remote computer system of a user (e.g., remote cloud server). Examples of remote computer systems include, but are not limited to, personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1001 via the network 1030.

Methods as described herein can be implemented by way of machine (e.g., computer processor) executable code stored on an electronic storage location of the computer system 1001, such as, for example, on the memory 1010 or electronic storage unit 1015. The machine executable or machine readable code can be provided in the form of software. During use, the code can be executed by the processor 1005. In some cases, the code can be retrieved from the storage unit 1015 and stored on the memory 1010 for ready access by the processor 1005. In some situations, the electronic storage unit 1015 can be precluded, and machine-executable instructions are stored on memory 1010.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1001, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, for example, shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include, but are not limited to, for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1001 can include or be in communication with an electronic display 1035 that comprises a user interface (UI) 1040 for providing, for example, an electronic output of identified gene fusions. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1005.

### EXAMPLES

### Example 1—Preparation of Immune Organoids From Tonsils and Other Lymphoid Tissues

Human tonsil cultures were developed with dissociated cells that reaggregated in culture. Table 1 below shows the characteristics of the tissue donors.

TABLE 1
Characteristics of tissue donors.

| | | race or | indication for | | recent influenza |
|---|---|---|---|---|---|
| Donor | sex | ethnicity | surgery | age | vaccination |
| IMD005 | F | Hispanic | ND* | 3 | ND |
| IMD006 | F | Caucasian | ND | 8 | ND |
| IMD008 | F | Hispanic | ND | 6 | ND |
| IMD009 | F | Hispanic | OSA** | 8 | ND |
| IMD010 | M | Caucasian | OSA, otitis media | 2 | ND |
| IMD012 | M | Hispanic | hypertrophy | 9 | yes |
| IMD013 | F | Hispanic | OSA, otitis media | 3 | ND |
| IMD014 | F | Caucasian | ND | 14 | yes |
| IMD019 | F | Hispanic | OSA | 7 | yes |
| IMD021 | M | Hispanic | tonsillitis | 5 | no |
| IMD022 | M | Hispanic | hypertrophy | 6 | no |
| IMD024 | M | Caucasian | OSA | 3 | ND |
| IMD027 | M | Caucasian | ND | 3 | no |
| IMD029 | M | Filipino | ND | 9 | yes |
| IMD030 | F | Caucasian | ND | 2 | no |
| IMD035 | F | Asian | hypertrophy | 3 | yes |
| IMD049 | M | Caucasian | OSA | 6 | ND |
| IMD058 | F | Caucasian | OSA | 3 | ND |
| IMD068 | M | Caucasian | ND | 5 | ND |
| IMD069 | M | Caucasian | OSA | 3 | ND |
| IMD070 | M | Hispanic | OSA | 6 | ND |
| IMD072 | M | Hispanic | OSA | 6 | ND |
| IMD073 | M | Caucasian | OSA | 5 | ND |
| IMD079 | M | Hispanic | velopharyngeal | 7 | ND |
| | | | insufficiency | | |
| IMD080 | M | Asian | OSA | 5 | ND |
| IMD081 | M | Caucasian | OSA | 7 | ND |
| IMD082 | M | Caucasian | sleep-disordered | 2 | ND |
| | | | breathing | | |
| IMD083 | M | Hispanic | OSA | 10 | yes |
| IMD086 | M | Caucasian | OSA | 8 | no |
| IMD087 | F | Caucasian | OSA | 10 | yes |
| IMD088 | F | Hispanic | OSA | 4 | no |
| IMD092 | F | Caucasian | OSA | 5 | no |
| IMD093 | M | Asian, | OSA | 2 | yes |
| | | Caucasian | | | |
| IMD095 | F | Caucasian | OSA | 4 | yes |
| IMD096 | M | Hispanic | OSA | 8 | yes |
| IMD098 | F | Caucasian | OSA | 5 | no |
| IMD099 | M | African | OSA | 5 | no |
| | | American | | | |
| IMD100 | M | Hispanic | OSA | 7 | yes |
| IMD102 | F | Hispanic | OSA | 9 | ND |
| IMD103 | M | Hispanic | OSA | 4 | yes |
| IMD104 | F | Caucasian | hypertrophy | 8 | yes |
| IMD107 | M | Hispanic | tonsillitis | 9 | yes |
| IMD108 | M | Caucasian | OSA | 4 | yes |
| IMD111 | M | Caucasian | OSA | 15 | yes |
| IMD113 | F | Caucasian | OSA | 9 | yes |
| IMD117 | M | Caucasian | OSA | 7 | no |
| IMD120 | F | Caucasian | OSA | 3 | no |
| IMD144 | F | Caucasian | OSA | 3 | yes |
| IMD145 | M | Hispanic | OSA | 3 | yes |
| IMD148 | M | Other | OSA, hypertrophy | 8 | no |
| IMD150 | F | Hispanic | OSA, hypertrophy | 7 | yes |
| IMD151 | M | Hispanic | OSA | 4 | no |
| IMD153 | F | Hispanic | OSA | 8 | yes |
| IMD163 | M | Asian, | OSA | 9 | yes |
| | | Caucasian | | | |
| IMD166 | F | Hispanic | OSA | 11 | yes |
| IMD167 | M | ND | OSA | 2 | yes |
| IMD170 | M | Caucasian | OSA | 7 | yes |
| VIP012 | F | caucasian | OSA | 33 | no |
| VIP013 | M | caucasian | OSA | 72 | ND |
| VIP017 | F | Hispanic | ND | 54 | ND |
| LN/spleen | M | ND | NA*** | 53 | ND |
| 629 | | | | | |
| LN/spleen | M | ND | NA | 64 | ND |
| 654 | | | | | |
| LN/spleen | M | ND | NA | 65 | ND |
| 655 | | | | | |
| LN/spleen | F | ND | NA | 75 | ND |
| 665 | | | | | |
| LN/spleen | F | ND | NA | 44 | ND |
| 680 | | | | | |
| LN/spleen | F | ND | NA | 59 | ND |
| 690 | | | | | |

*ND no data
**OSA obstructive sleep apnea
***NA not applicable

Whole tonsils from 150 consented individuals undergoing surgery for obstructive sleep apnea, hypertrophy or recurrent tonsillitis were collected in accordance with the Stanford University Institutional Review Board (IRB). Ethics approval was granted by the Stanford University IRB (protocols 30837 and 47690). Written informed consent was obtained from adult participants and from the legal guardians of children aged 0-17 years; written informed assent was also obtained from children aged 7 years and older. In this cohort, the participants were children aged 2-17 years (n=57) and adults (n=3) who had surgery for obstructive sleep apnea and/or hypertrophy, and overall, tonsil tissue was typically healthy. Whole tonsils were collected in saline after surgery and then immersed in an antimicrobial bath of Ham's F12 medium (Gibco) containing Normocin (InvivoGen), penicillin and streptomycin for 1 h at 4° C. for decontamination of the tissue. Tonsils were then briefly rinsed with PBS and processed as needed for culturing (see below).

Donor lung lymph nodes and spleens were provided by the Gift of Hope Organ and Tissue Donor Network to the University of Chicago. These tissues were determined to have IRB Exempt Status by the University of Chicago IRB. Only de-identified demographic information was obtained.

For cryopreservation of tonsil cells, tissue was dissected into roughly 5 mm×5 mm×5 mm pieces and manually disrupted into a suspension by processing through a 100-μm strainer with a syringe plunger. Enzymatic dissociation was not necessary and did not improve the response to LAIV from cryopreserved cells. Tissue debris was reduced by Ficoll density gradient separation, although this step was not required for tonsil organoid development. After washing with complete medium (RPMI with glutamax, 10% FBS, 1× nonessential amino acids, 1× sodium pyruvate, 1× penicillin-streptomycin, 1× Normocin (InvivoGen) and 1× insulin/selenium/transferrin cocktail (Gibco)), cells were enumerated and frozen into aliquots in FBS+10% DMSO. Frozen cells were stored at −140° C. until use.

For lung-draining lymph nodes and spleen samples, tissues were collected in saline or Hank's Balanced Salt Solution, diced into small pieces and pressed through nylon mesh (Nitex) to break up the tissue. Cells were isolated with Ficoll density gradient separation, washed, enumerated and frozen in FBS+10% DMSO. Frozen samples were stored in liquid nitrogen until use.

For culture of cryopreserved cells, aliquots were thawed into complete medium, enumerated and resuspended to 6×10⁷ cells per ml for larger cultures or 2×10⁷ cells per ml for smaller cultures. Cells were plated, 100 μl per well, into permeable (0.4-μm pore size) membranes (24-well size PTFE or polycarbonate membranes in standard 12-well plates or 96-well polycarbonate membrane plates with single-well receiver trays; Corning or Millipore), with the lower chamber consisting of complete medium (1 ml for 12-well plates, 200 μl for 96-well plates) supplemented with 1 μg ml−1 of recombinant human B cell-activating factor (BAFF; BioLegend). Adding a small amount of BAFF improved total B cell survival (and thus increased overall cell recovery) but was not a requirement for plasmablast differentiation or antibody secretion.

LAIV (1 μl per well, equivalent of 1.6×10⁴ to 1.6×10⁵ fluorescent focus units per strain; FluMist Quadrivalent, Medimmune), wild-type influenza virus (A/California/07/2009 pandemic strain; a gift from H. Greenberg and X.-S. He), MMR vaccine (5 μl per culture; Merck), R-phycoerythrin (1 μg per culture; Thermo Fisher), rabies vaccine (10 μl per culture; Imovax, Sanofi Pasteur) or Ad5-vectored SARS-COV-2 vaccine candidate (1×10⁸ infectious units per culture; Vaxart) was then added directly to the cell-containing portion of the culture setup. For adjuvant testing, alum (0.01%; InvivoGen, sold as a 2% stock wet gel with 9-11 mg ml−1 stock aluminum content) or imiquimod (2.5 μg ml−1; InvivoGen) was added directly into the culture immediately after antigen addition. Cultures were incubated at 37° C., 5% CO2 with humidity and supplemented with additional medium to the lower wells as necessary. Hypoxic conditions (5% O2) were tested for B cell differentiation and antibody secretion and were not significantly different from cultures maintained at standard incubator oxygen levels (17-21%).

For the Ad5-vectored vaccine candidates, recombinant adenoviral constructs were produced using the publicly available SARS-COV-2 DNA sequence (GenBank accession no. MN908947.3). Spike and nucleocapsid protein sequences were synthesized and codon optimized for expression in human cells and cloned into the E1 region as previously described. The same vector backbone has been used previously in clinical trials for oral recombinant adenovirus tablets. The Ad-S adenoviral vector contains a spike protein under the human cytomegalovirus (CMV) promoter. The Ad-SN vector contains spike under the CMV promoter and nucleocapsid under the human beta-actin promoter. The recombinant Ad-S1N vector uses a fusion sequence combining the S1 region of the SARS-COV-2 spike gene (including the native furin site between S1 and S2) with the full-length SARS-COV-2 nucleocapsid gene. All vaccine candidates were purified by cesium chloride density centrifugation and provided in a liquid form for cell culture experiments.

For organoid preparation, frozen single-cell suspensions from tonsil tissues were thawed and plated at high density into the wells of permeable membrane plates (e.g., transwells) along with the antigen of interest. After several days in culture, reaggregated regions of clustered cells were visible as can be seen in FIG. 1A. The cell composition of the reaggregated cultures were assessed after 7 days in the presence or absence of antigen and the optimized conditions sustained appropriate tonsil cell composition, as can be seen in FIG. 1B.

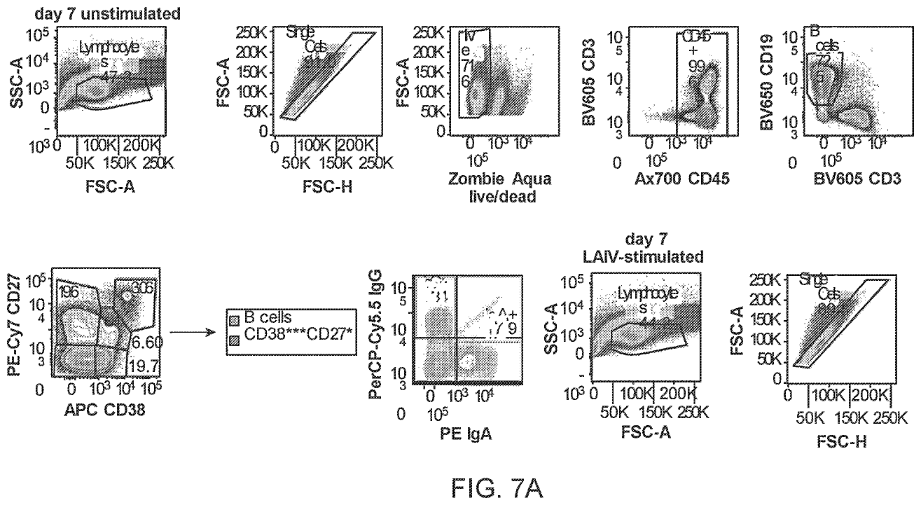

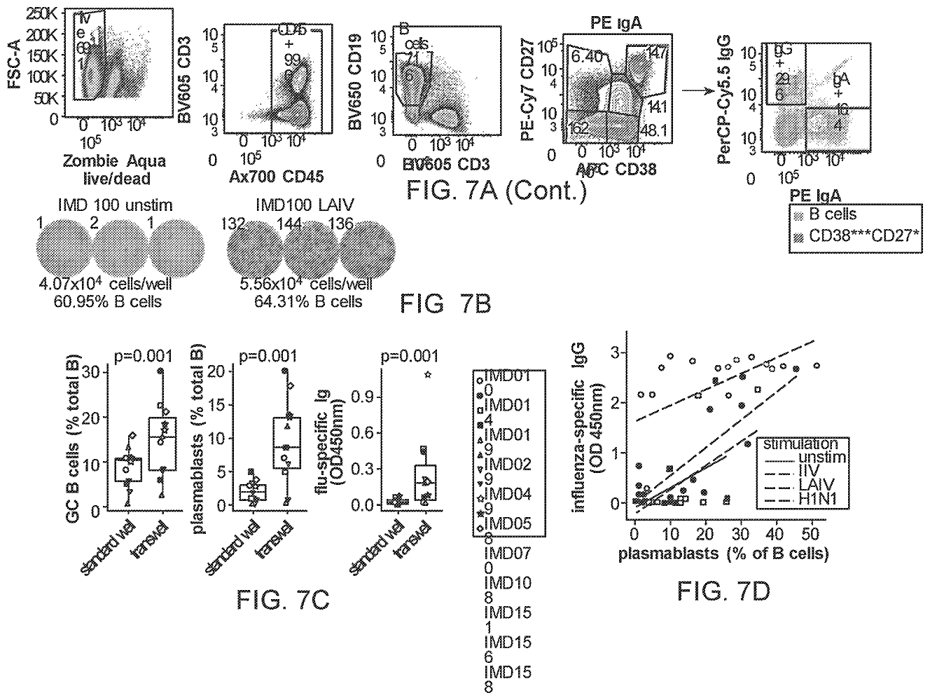

Influenza vaccines and viruses were used as model antigens since much is already known about the features of the human influenza response in vivo. Upon stimulation with live attenuated influenza vaccine (LAIV), there were notable increases in B cell differentiations and a more structures culture morphology developed, suggesting additional activity in response to this immunogen. The capacity of organoid cultures to support B cell maturation and function upon LAIV stimulation was then measured by staining the cultures as can be seen in FIG. 7A.

Flow cytometry. Organoids were harvested from the upper portion of the permeable membranes by rinsing the membranes with PBS. Cells were washed with FACS buffer (PBS+0.1% BSA, 0.05% sodium azide and 2 mM EDTA) and stained at 4° C. with the following anti-human antibodies in the presence of Fc block and live/dead Aqua Zombie stain, all from BioLegend unless otherwise noted: FITC CD138 (1/100), FITC CD116 (1/100), FITC CD21 (1/50), FITC or Ax488 CXCR5 (1/33), PerCP-Cy5.5 CD8 (1/100), PerCP-Cy5.5 CD33 (1/100), PE CD19 (1/100), PE CD56 (1/100), PE gamma-delta TCR (1/100), PE-Cy7 CD27 (1/100), PE-Cy7 CD123 (1/50), PE-Cy7 CD8 (1/100), APC CD38 (1/200), APC HLA-DR (1/100), APC CD27 (1/200), Ax700 CD45 (1/100), Ax700 CD14 (1/100), APC-Cy7 IgD (1/50), APC-Cy7 CD16 (1/100), APC-Cy7 CD45RA (1/100), Pacific Blue HLA-DR (1/100), Pacific Blue PD-1 (1/50), BV605 CD3 (1/100), BV650 CD4 (1/100), BV650 CD19 (1/100), BUV395 IgM (1/20; BD Biosciences) and BUV395 CD45RA (1/20; BD Biosciences).

For AID staining, after surface staining, the cells were fixed and permeabilized (eBioscience) and stained intracellularly with biotinylated anti-AID antibody (1/100; clone mAID-2; eBioscience) followed by PE-streptavidin (eBioscience). A no-AID antibody control was used to discriminate positive signal. All analyzer data were collected on BD LSRII instruments and analyzed using FlowJo (TreeStar).

It was found that after 7 days, plasmablast frequencies were significantly increased compared to unstimulated controls (FIG. 1C; n=15; P=0.0002). Thirteen of the fifteen donors tested, ranging in age from 2-14 years, produced influenza-specific IgG antibodies in only the LAIV-stimulated samples (P<0.0001). Of the two exceptions, one donor had a high response without stimulation and the other was a true nonresponder. The latter donor (a 3-year-old) may have been naive for influenza infection and had no reported influenza vaccination. The production of specific antibody was not a generic effect of stimulation, as total (that is, antigen agnostic) IgG was reduced by LAIV (FIG. 1D). Enzyme-linked immune absorbent spot (ELISpot) analysis was performed on day-7 cultures from five donors and influenza-specific antibody-secreting cells (ASCs) ranging from 0.1-1.5% of total B cells was found (FIG. 1E; representative ELISpot data in FIG. 7B). Almost no influenza-specific ASCs were identified in unstimulated tonsil cultures (<0.02% of total B cells in all donors). The Transwell organoid culture strategy was superior to flat −bottom tissue culture plates of similar surface area for growing tonsil mononuclear cells (FIG. 7C).

Antibody detection by ELISA. For detection of influenza-specific antibodies, ELISA plates (Costar) were coated with 0.1 μg per well of season-matched Fluzone Quadrivalent inactivated influenza vaccine (based on reported total HA content from the manufacturer; Sanofi) to act as the capture antigen. For A/California HA antibody detection, recombinantly expressed soluble HA trimers were used in place of the inactivated vaccine as the capture antigen. Diluted (1:20 or 1:50) culture supernatants were added to coated, blocked plates. A human pan-influenza monoclonal IgG antibody (H1N13-M; Alpha Diagnostics) was used as a standard to estimate specific antibody concentration for experiments where antibody concentration is quantitatively defined. Horseradish peroxidase-conjugated anti-human secondary antibodies to either IgM/IgG/IgA (Sigma) or Fc-IgG alone (Bethyl; adsorbed for other isotypes) were used to detect bound antibodies. Plates were developed with TMB substrate solution (Thermo Scientific), quenched with sulfuric acid and read at 450 nm. Neutralization experiments were performed by Monogram Biosciences with a pseudovirus neutralization assay with HA matching the vaccine antigen strains.

For detection of total IgG, culture supernatants were diluted at 1:500 and assayed by ELISA (Thermo Scientific) following manufacturer's instructions. For detection of MMR-specific IgG, culture supernatants were tested by ELISA (Abcam) following the manufacturer's instructions. Supernatants were diluted at 1:2.5 with the provided sample diluent and serial twofold dilutions out to 1:40 confirmed signal specificity (data not shown). To measure rabies nucleoprotein-specific IgM and IgG, supernatants were diluted at 1:5 with specific IgM and IgG detection kits (Alpha Diagnostic).

Antibody detection by protein microarray. Organoid cultures stimulated with recombinant adenoviral vectors with SARS-COV-2 sequences were tested for specific antibody production using a protein microarray. The technology has been previously described for the detection of influenza-specific antibodies and was recently adapted to include commercially available SARS-COV-2 proteins. The SARS-COV-2 proteins are commercially available from Sino Biological. Briefly, day-14 culture supernatants were diluted 1:1 with blocking buffer and incubated for 30 min. Then, diluted samples were added to the microarray for overnight hybridization. The array was washed three times with Tris-buffered saline containing Tween20, then treated with Qdot-conjugated anti-IgM, IgG or IgA secondary antibodies for 2 h. After another three washes, the array was dried and read, with signal intensities representing relative antibody quantities bound to each protein spot.

Also tested was whether the organoid culture strategy could support human lung-draining lymph node and spleen samples (FIG. 1F). These organoids responded to both LAIV and wild-type A/California H1N1 influenza virus by producing influenza-specific antibodies at levels substantially above those of control cultures and equivalent to tonsil organoids.

### Example 2: Longitudinal Analysis of Tonsil Cultures in Response to LAIV

Given the evidence of B cell maturation and antibody secretion in organoid cultures stimulated with LAIV, longitudinal analyses were performed to observe the differentiation process over time. CD38 and CD27 expression patterns on B cells from LAIV-stimulated cultures clearly evolved (FIG. 2A), indicating an early transition to a pre-GC phenotype (CD38+CD27−), and in two of three donors, this was followed by plasmablast differentiation (FIG. 2B). The frequency of plasmablasts typically correlated with the total amount of specific antibodies produced (FIG. 7d). Changes in T cell phenotypes were also observed, including recovery of TFH (CXCR5+CD4+) cells during culture (in both unstimulated and LAIV-stimulated organoids), as well as transient CD4+ and CD8+ T cell activation at day 7 in LAIV-stimulated cultures (FIG. 2C). Organoids prepared from only one of the over 50 donors tested (IMD012) did not produce influenza-specific antibodies (FIG. 2D). With four additional donors, it was confirmed that influenza-specific antibodies are produced with the expected kinetics (between days 5 and 10). The ability of these cultures to produce neutralizing antibodies was gauged; in the two strains tested (of the four LAIV strains), vaccine-stimulated culture supernatants showed neutralizing capabilities, while unstimulated controls did not (FIG. 2E).

The T cell response in HLA-A2+ donors was also assessed using class I tetramers targeting the immunodominant influenza M1 specificity (FIG. 2F). Three of the four donors tested showed a two-to ten-fold expansion of M1-specific CD8+ T cells by day 7 after stimulation with LAIV. The one nonresponder had fewer M1-specific CD8+ T cells in their starting repertoire compared to the other donors, which may explain their poor CD8+ T cell response.

Tetramer staining. HLA-A2 tetramers were prepared as previously described, with ultraviolet (UV)-sensitive peptide cleavage and exchange for the A2 immunodominant influenza M1 peptide (GILGFVFTL) or an irrelevant CMV pp65 peptide (NLVPMVATV) as a control; tetramers were prepared from the peptide-exchanged monomers by conjugation to PE-streptavidin and APC-streptavidin, respectively (eBioscience). For staining, HLA-A2 donor organoids were harvested, washed with FACS buffer and stained for 1 h at 4° C. with tetramers (0.5 μg of monomer per test) in the presence of Fc block. During the last 30 min of tetramer staining, lineage-defining antibodies were added, and samples were washed with FACS buffer. For analysis, influenza M1 tetramer-positive CD8+ T cells were identified as single-positive T cells for the influenza tetramer (negative for CMV tetramer) and with staining above a no-influenza-tetramer staining control.

### Example 3: Spatial Organization of Tonsil Organoids

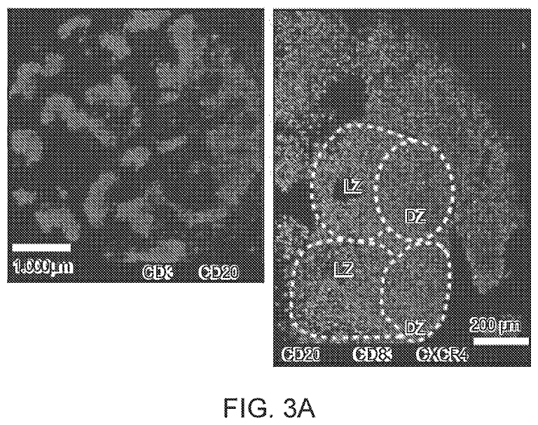

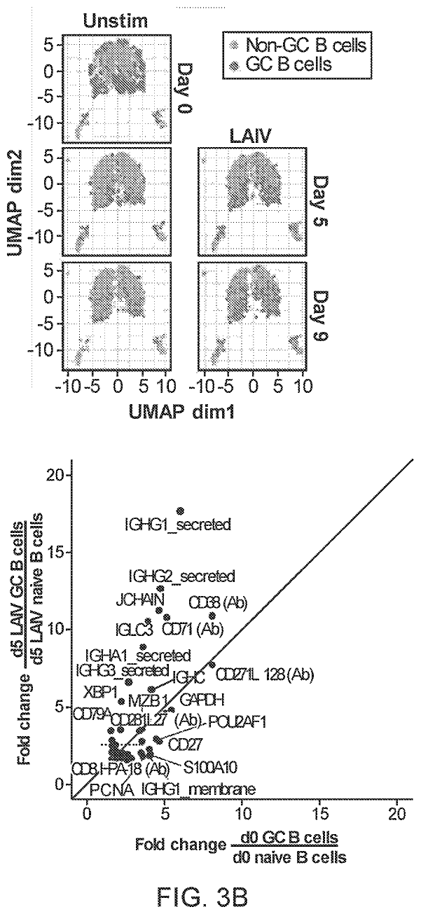

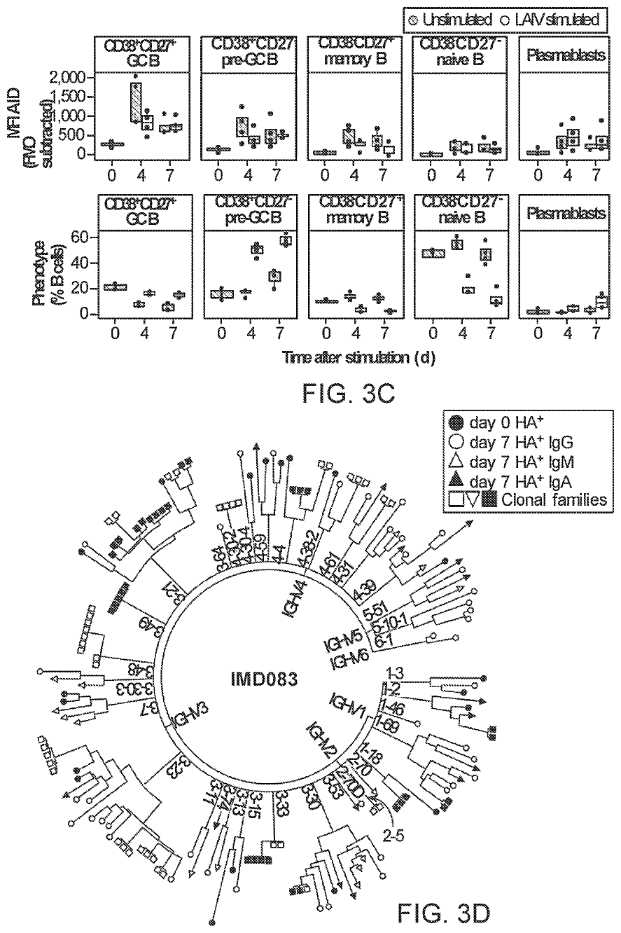

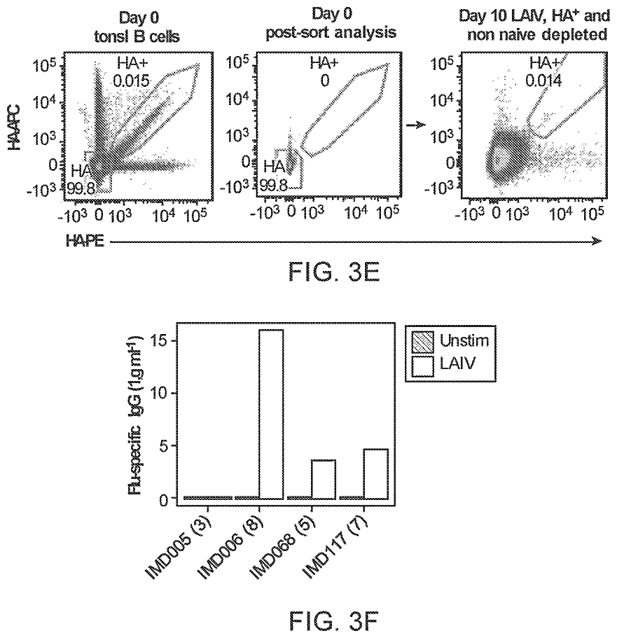

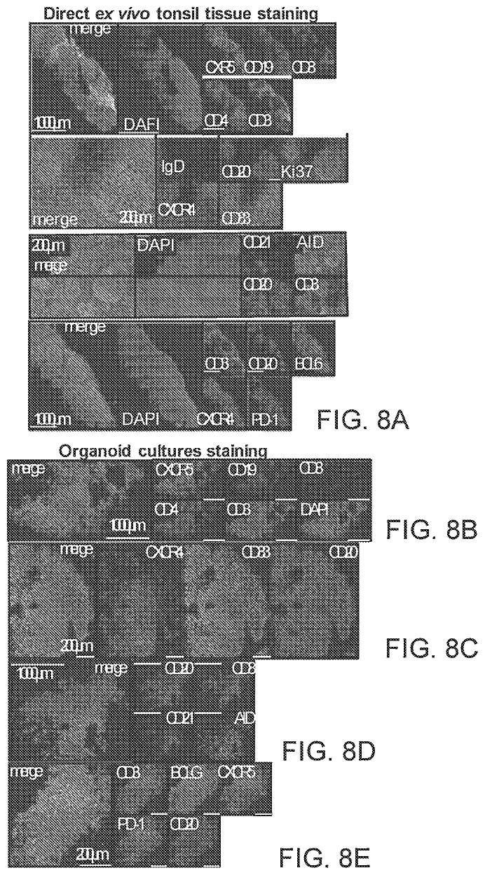

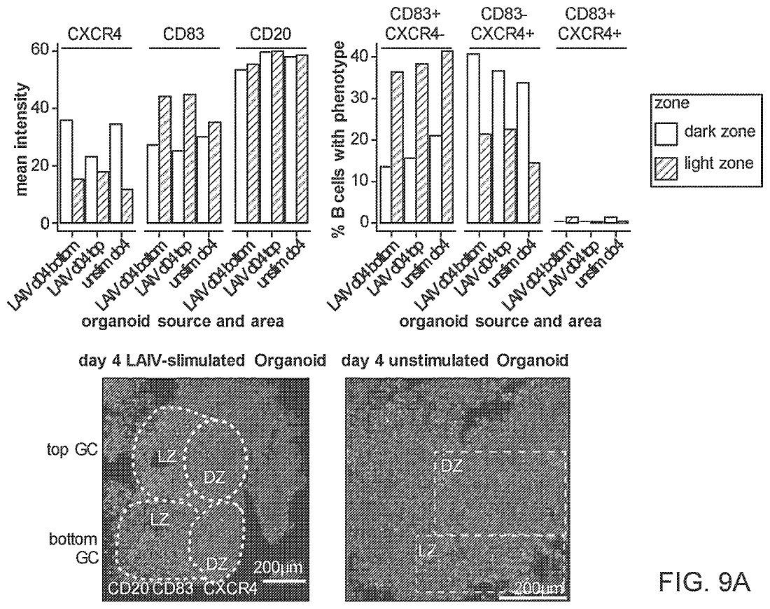

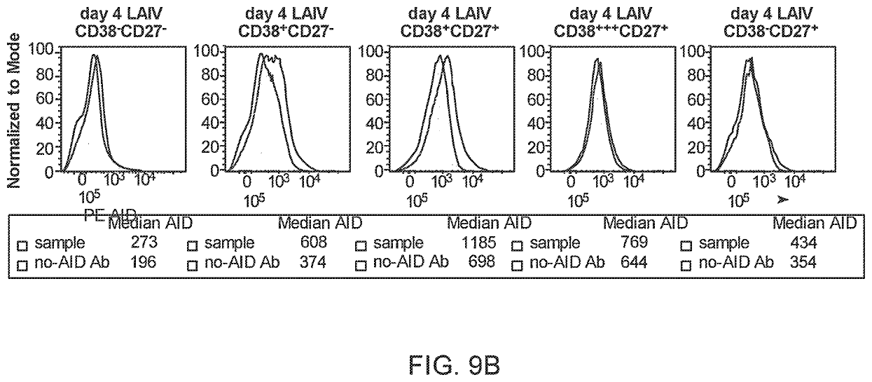

During an adaptive immune response, peripheral lymphoid organs like the tonsils, lymph nodes and spleen develop GCs. B cells congregate in the GC area, whereas T cells are largely in outlying areas. In the organoid cultures, GC-like structures with distinct B cell-and T cell-rich aggregates in both LAIV-stimulated and unstimulated cultures were found, starting around 48 h after culture initiation. These progressed to well-defined clusters by days 4-7, particularly in LAIV-treated cultures (FIG. 3A and FIG. 8). The embedded, frozen sections of three donors by fluorescent confocal microscopy were analyzed and evidence of light and dark zone organization was found, as detected by separation of CD83 and CXCR4 staining B cells, respectively, which are characteristic of GCs (FIG. 3A; representative images FIGS. 8A-8E and FIG. 9A).

Immunofluorescence. Immunofluorescence microscopy samples were prepared from frozen tonsil cells stimulated with LAIV and harvested 4 or 7 d after stimulation. Permeable membrane inserts containing organoids were gently immersed in PBS, fixed with 4% paraformaldehyde in PBS for 30 min at 4° C. and washed three times with water. Cultures were kept at room temperature and incubated for 20 min in increasing concentrations of warmed (37° C.) OCT Compound (Fisher) diluted in PBS at 25%, 50% and 75% (vol/vol), with a final incubation in pure OCT. Samples were snap frozen on dry ice, inserts were removed with forceps, and samples were embedded in an additional layer of OCT and frozen. Embedded samples were sectioned at 25 μm and adhered to poly-l-lysine-coated coverslips. Sections were dried for 4 min in a dehumidified chamber and permeabilized in acetone for 10 min at room temperature. Rehydration was performed in stain buffer (1% BSA, 1% normal goat serum and 0.01% sodium azide in PBS) for 30 min. Sections were stained first with primary unconjugated antibodies at room temperature for 3 h. Secondary antibody staining was performed for 1 h at room temperature followed by primary direct conjugate antibodies for 3 h at room temperature.

Primary antibodies. Immunofluorescence staining was performed using the following antibodies at the stated concentrations: BioLegend: CD3-BV421 (SK7; 1:25), CD4-AF594 (RPA-T4; 1:50), CD19-AF647 (SJ25C1; 1:25), CXCR4 (12G5; 1:100), CD21-FITC (Bu32; 1:100), PD-1-AF488 (NAT105; 1:100), BCL-6 (IG191E/A8; 1:50); Sigma-Aldrich: CXCR5 (Polyclonal; 1:100), CD83 (Polyclonal; 1:100); Thermo Fisher: AID (ZA001; 1:100), CD8-AF488 (AMC908; 1:100), CD20-eFluor 615 (L26; 1:100), CD20-eFluor 660 (L26; 1:100); BD Biosciences: Ki67-BV421 (B56; 1:5); Fisher Science: IgD-AF488 (IgD26; 1:10).

Secondary antibodies. The following secondary antibodies from Thermo Fisher and their dilutions were used: goat anti-mouse IgG (H+L) AF Plus 555 (1:200), goat anti-rabbit IgG (H+L) AF Plus 555 (1:200), goat anti-rabbit IgG (H+L) AF Plus 594 (1:200).

After collecting imaging data from antibody markers, slides were stained with 1 μg ml−1 DAPI to visualize nuclear staining. Imaging was performed on an Inverted Zeiss LSM 880 confocal instrument using ×25 magnification. Sample z-stacks were taken at 2-μm slices. DAPI was stimulated at 405 nm, FITC/Alexa Fluor 488 at 488 nm, Alexa Fluor 555 at 561 nm and Alexa Fluor 594 at 594 nm. Images were processed in ImageJ (version 2.0.0). Tile stitching was performed with the Grid/Collection stitching tool using positions from files in the order defined by the image metadata. The fusion method used was Linear Blending, with Computer Overlap and Ignore Calibration (all other parameters were set to default). After despeckling, z-stacks were merged using ZProjection by Maximum Intensity and contrast adjusted to better present structure, and channels were stacked to RGB and a scale bar added.

To quantify the fraction of CXCR4- and CD83-expressing cells in GC areas, GCs were cropped into two portions that resembled light and dark zone-like regions as shown on the imaging figure. CD20-, CD83- and CXCR4-positive cells were manually counted (multipoint tool in ImageJ) in addition to double- and triple-positive cells. CD83+CXCR4−CD20+ and CD83−CXCR4+CD20+ cells in each region were calculated and presented as a proportion of the total CD20+ population from the same area. To determine the mean intensities of marker expressions in light and dark zone regions, images were converted to RGB using ‘stack to RGB’ and then ‘color histogram’ was performed on each channel using ImageJ, which displays a histogram of the intensity values. The mean of each channel was used to compare the average marker expression between different regions of equivalent size.

ELISpot. ASCs were detected using an ELISpot protocol. Cultures that were either stimulated for 7 d with LAIV or left unstimulated were resuspended and enumerated, then plated on inactivated influenza vaccine-coated and blocked 96-well PVDF membrane plates (Millipore). Each sample was plated with three, threefold dilutions in triplicate, and total live-cell counts ranged from 2.22×10⁴ to 1.07×10⁵ cells per well at a 1:9 dilution, which was used for enumeration of ASCs. Cells were incubated on these membranes, undisturbed for 5 h at 37° C. Plates were then washed and treated with horseradish peroxidase-conjugated anti-IgG/IgA/IgM secondary antibody. After incubation overnight at 4° C., plates were washed and developed with AEC substrate (BD), washed 20 times with water, dried, and spots were enumerated. The frequency of ASCs out of total B cells was determined from B cell flow cytometry data analysis and the direct cell enumeration counts.

### Example 4: Expression Profile Characterization

A single-cell RNA-seq analysis was performed along with DNA-barcoded antibodies using the BD Rhapsody platform to characterize the expression profiles of GC-phenotype B cells in organoid cultures. Dimensionality reduction (uniform manifold approximation and projection (UMAP)) was used to analyze GC B cells across different time points (FIG. 3B). Overall, GC B cells (CD38+CD27+) from the different days and time points clustered together, showing that their expression profiles were similar. The top overexpressed genes in GC B cells (compared to naive B cells) at day 5 of LAIV-stimulated cultures were related to antibody secretion (immunoglobulin heavy chains, light chains and J chain) and key B cell differentiation, proliferation and B cell receptor (BCR) signaling genes (XBP1, POU2AF1, S100A10 and PCNA). These were also the top differentially expressed genes between direct ex vivo GC and naive B cells. These data show that the cultures can functionally respond to antigen-specific stimulation, that light and dark zone phenotype B cells separate into distinct areas (FIG. 3A and FIG. 9A) and that B cells with a GC phenotype have similar transcriptional profiles on day 0 and day 5 after culture (FIG. 3B), which collectively support the conclusion that these cultures can be considered organoids.

Single-cell RNA-seq. Cells from either day-0 tonsils (processed, cryopreserved and thawed) or organoids (from day 5 and day 9 after stimulation with LAIV or left unstimulated) were stained with a mixture of fluorophore-conjugated antibodies to enable sorting for CD45+CD19+CD3− B cells and for sequencing detection of DNA-tagged antibodies (CD20 clone L27, CD19 clone SJ25C1, CD71 clone L01.1, IgM clone G20-127, IgA clone A59, CD161 clone HP-3G10, CD27 clone L128, CD38 clone HIT2, IgD clone IA6-2, IgG clone G18-145, CD83 clone HB15E, CXCR4 clone 12G5, TCR Vg9 clone B3, CD3 UCHT1, CD4 clone SK3, CD8 clone RPA-T8 and CXCR5 clone RF8B2). A cocktail of DNA-tagged antibodies was used to allow manual gating on B cells (CD19+CD3−) and B cell subsets (CD27 and CD38) to identify GC B cells and naive B cells. The sorted cells were tagged with sample barcodes to enable pooling. The cells were loaded and captured from the pooled sample using the BD Rhapsody pipeline following the manufacturer's instructions to prepare the libraries, using the targeted human immune gene panel for amplification. Libraries were sequenced using Illumina Novaseq platforms and the resulting data processed using the Rhapsody analysis pipeline. Using SeqGeq (BD) software, individual samples were debarcoded, and B cell subsets were gated based on DNA-barcoded antibodies for CD3, CD19, CD27 and CD38. Individual populations were then exported along with their gene expression profiles for analyses. UMAP dimensionality reduction was run using the R package ‘umap’ on gene (and not DNA-tagged antibody) expression profiles. For fold change expression analyses, genes that were overexpressed in GC B cells by at least 1.5-fold were plotted.

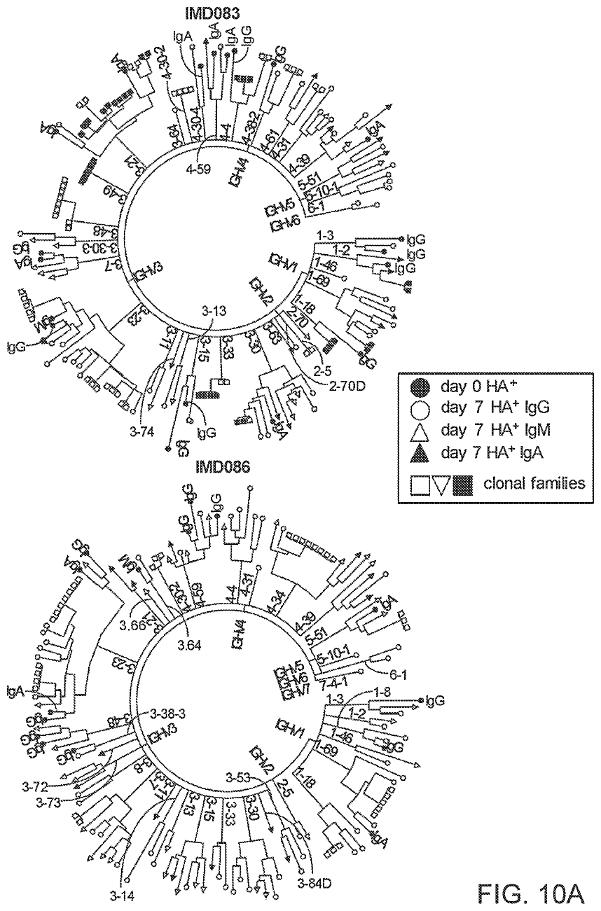

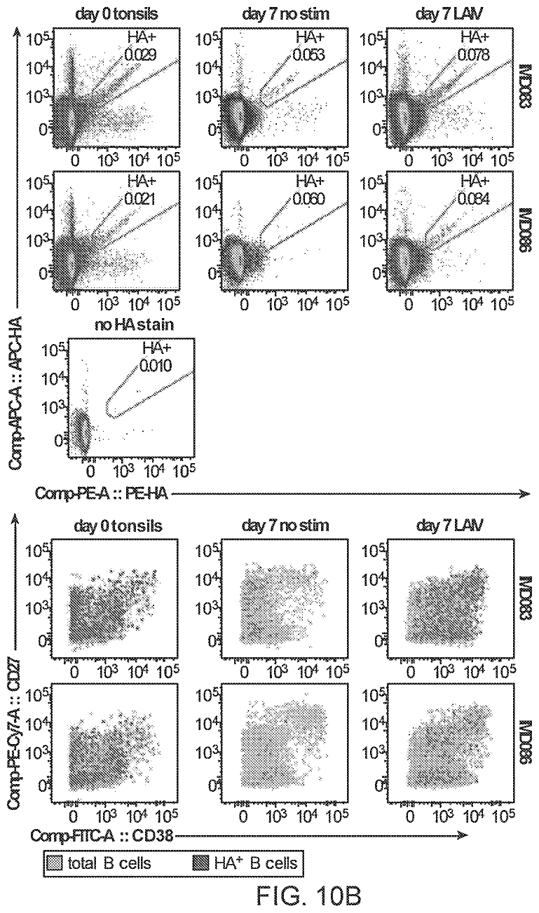

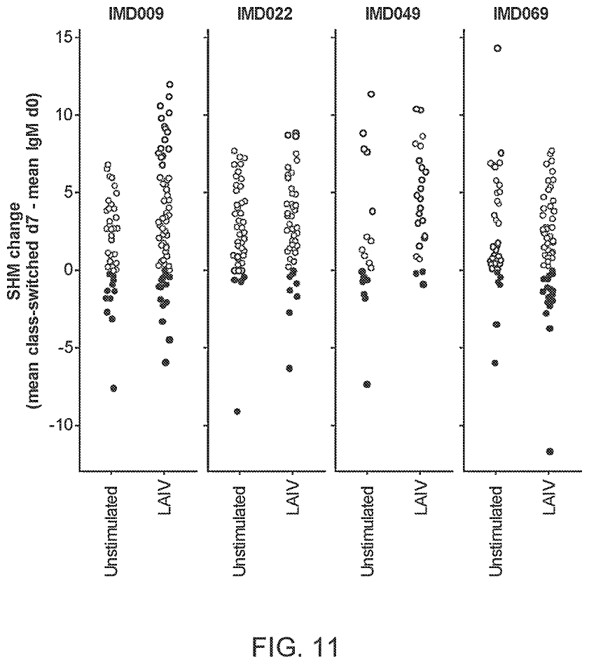

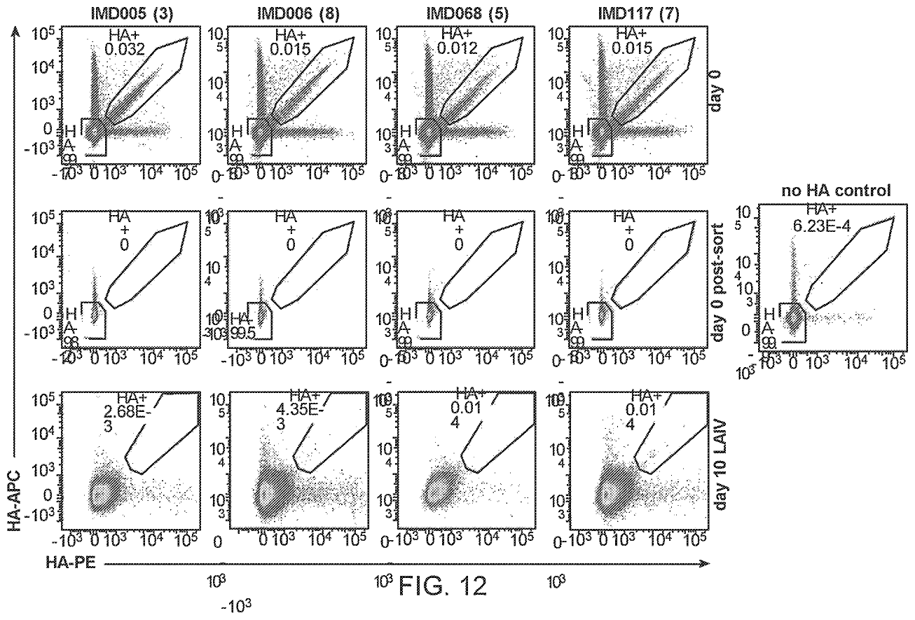

Diversity and maturation of hemagglutinin-specific B cells. Next, it was examined whether activation-induced cytidine deaminase (AID), which is required for both somatic hypermutation and class switching, was expressed in these cultures. AID protein levels (FIG. 3C; see representative staining in FIG. 9B) were significantly increased in pre-GC and GC B cells in 4-day-old organoid cultures (prevalence shown in FIG. 3C). BCRs were then single-cell sequenced from high-affinity A/California 2009 H1N1 hem-agglutinin (HA)-specific plasmablasts (and other activated B cells) using a HA trimer construct37 on day-0 and day-7 LAIV-stimulated cultures to evaluate affinity maturation and isotype switching. As previously reported in peripheral blood plasmablasts of influenza-vaccinated volunteers38, immunoglobulin heavy-chain variable gene cluster (IGHV) 3 and IGHV4 dominated the response (FIG. 3D; FIG. 10A-10B for complete dataset). HA-specific BCRs from LAIV-stimulated organoids were quite diverse in both gene usage and isotype; numerous oligoclonal expansions were seen at day 7, but these clones were distinct from those detected in day-0 high-affinity HA+ B cells. Clonally related BCR families were also found in LAIV-stimulated organoids, and in one of two donors, clonal families with single amino acid changes were found, showing diversification within HA-specific lineages. Using bulk BCR sequencing of total non-naive B cells, lineages that began as the IgM isotype and class switched during organoid culture (mostly to IgG1 and IgA1) were found, usually with additional mutations, by day 7 (FIG. 11).

B cell receptor sequencing. For isotype-switching analysis, tonsil cells from day-0 or day-7 organoids were harvested, washed with FACS buffer, and stained with a cocktail of lineage-defining antibodies as above in the presence of Fc block, then bulk sorted using a FACS Fusion or Aria II instrument. Bulk sequencing of immunoglobulin heavy-chain gene rearrangements for isotype-switching analysis was carried out as previously reported. Briefly, RNA was isolated from sorted (memory CD38−CD27+, GC CD38+CD27+ and plasmablast CD38+++CD27+) cells using Trizol (Thermo Fisher) and reverse transcribed to cDNA using Superscript II (Life Technologies) primed with random hexamer primers. Amplicons from IgM, IgD, IgG, IgA and IgE were PCR amplified in separate reactions, using IGHV framework region 1 primers and isotype primers in the first constant region exon, modified to contain partial Illumina linker sequences, sample barcode sequences and randomized nucleotides to ensure sequence diversity in the initial cycles of sequencing. A second PCR was carried out to complete the Illumina linker sequences before amplicon quantification, pooling, gel extraction (Qiagen) and sequencing on an Illumina MiSeq instrument with 600-cycle kits as 2×300 paired-end reads. Bulk BCR heavy-chain sequences were analyzed with an in-house developed pipeline, based on IgBLAST for V, D and J gene segment alignment and CDR-H3 parsing. Clonally related sequences in the bulk sequencing data were identified based on shared usage of IGHV and IGHJ genes, equal CDR-H3 length and single-linkage clustering of CDR-H3 nucleotide sequences at a 90% identity threshold.

For analyses of somatic hypermutation and A/California 2009 HA-specific B cells, tonsil cells from day-0 or day-7 organoids were harvested, washed with FACS buffer, then treated with 2 μg per sample (4 μg ml−1) of biotinylated recombinant A/California influenza HA1 hemagglutinin (Y98F mutant; a gift from B. Graham and the Vaccine Research Center) in the presence of Fc block, then washed and stained with 0.2 μg ml−1 of fluorescently labeled streptavidin and a cocktail of lineage-defining antibodies.

For single-cell sorting, HA+ B cells of GC or plasmablast phenotype (CD38+CD27+ or CD38+++CD27+, respectively) were sorted. Single-cell antibody sequencing was performed as previously described. After single-cell sorting into 96-well plates, cDNAs were labeled with well-specific barcode oligonucleotides and pooled by plate, followed by gene-specific PCR and library preparation with previously reported primer sequences65. Libraries were sequenced with Illumina MiSeq 2×300 paired-end sequencing. Sequence analysis was performed as previously described65. Briefly, fastq generation and plate demultiplexing were completed using the onboard MiSeq Generate FASTQ workflow. After quality filtering, paired reads were stitched, separated by well ID and consensus sequences determined by clustering well ID reads into operational taxonomic units68. Consensus operational taxonomic unit sequences were analyzed with version 1.5.7.1 of IMGT HighV-QUEST69. For single-cell data, clonal families were defined by the same V and J gene usage and at least 70% amino acid identity in the CDR3 locus for both heavy and light chains. A caveat of single-cell HA-specific B cell sequencing is that the most vigorously responding B cells may have reduced surface immunoglobulin expression as they convert to antibody secretion, and so it is possible that the highest ASCs could not be captured during HA-specific B cell sorting.

Bulk IgH sequencing and analysis was performed using MIDCIRS as previously described. Sequencing was performed on an Illumina MiSeq with the v3, 600-cycle kit. mRNA molecules were tagged with 12N randomized molecular identifiers (MIDs) during reverse transcription. Reads with the same MID were grouped together and then further clustered into subgroups based on 85% sequence similarity to separate distinct mRNA molecules that were tagged with the same MID. Consensus sequences were then formed from the MID subgroups to average out PCR and sequencing errors and mitigate amplification and sequencing bias. Clonal lineages were defined using single-linkage clustering on the consensus sequences with the same criteria as above. The size of the clonal lineage refers to the total number of consensus sequences, or mRNA molecules, that make up the lineage, while the diversity of the lineage refers to the number of unique consensus sequences within the lineage.

To infer HA specificity of total, unsorted B cells from day-7 tonsil cultures, all FACS-sorted, HA-specific B cell sequences were first grouped together (n=20,977). Then, all nucleotide sequences were translated to amino acid sequences, and each day-7 sequence was aligned to the pool of HA+ sequences to find the minimum distance to the nearest HA-specific sequence. If any sequence within a clonal lineage was found to be an exact match or one substitution away from a known HA-specific sequence, the lineage was labeled ‘HA-inferred’. Clonal lineages were then split into HA-inferred and non-A/California HA for further analysis.

### Example 5: Affinity Maturation in Tonsil Organoids

Affinity maturation is another key function of GCs. To evaluate this, depletion experiments using FACS to eliminate preexisting high-affinity HA+ B cells and also any non-naive B cells from the tonsil cell pool were performed. Cultures were then prepared from the depleted cells, stimulated with LAIV, and stained again on day 10 to assess the development of new high-affinity HA+ B cells (FIG. 3E and FIG. 12). In three of four donors tested, HA+ B cells reappeared in depleted cultures and influenza-specific secreted antibodies were detectable (FIG. 3F), demonstrating that affinity maturation can be directly observed in this system.

Cell depletion experiments. Thawed tonsil cells were stained and bulk sorted into culture medium using a FACS Aria II or Fusion (BD). Sorting experiments involved separating individual cell subsets using the following markers: myeloid cells and DCs (CD45+CD3−CD19−HLA-DR+, CD116+ or CD33+), pDCs (CD4 5+CD3−CD19−HLA-DR+CD123+), Treg cells (CD45+CD3+CD19−CD4+CD25+CD127lo), total B cells (CD45+CD3−CD19+), non-naive B cells (CD38+ and/or CD27+ IgM− and/or IgD−), naive B cells (CD38−CD27−IgM+IgD+), pre-GC B cells (CD38+CD27−), GC B cells (CD38+CD27+), memory B cells (CD38−CD27+) and plasmablasts/plasma cells (CD38+++CD27+). Cell types were depleted by FACS from day-0 tonsil cells and cultured with LAIV for 7 d. As controls, depleted cultures were reconstituted with the cell type that was originally sorted and plated at the same cell density as the depleted cultures. For depletion of HA-specific B cells, tonsil samples were stained as described in ‘BCR sequencing’ and HA-specific B cells were defined as CD45+CD19+CD3−HA+. A post-sort analysis was used to ensure depletion purity.

Statistical analysis. All statistical analyses were performed in R. For comparing paired samples (frequencies of plasmablasts and antibody secretion in unstimulated versus LAIV-stimulated cultures, standard versus Transwell responses, unstimulated versus MMR-stimulated cultures, somatic hypermutation analyses, studying the effect of CD4+ cell depletion on plasmablast differentiation and specific antibody secretion), paired Wilcoxon signed-rank tests (two-sided) were performed. To analyze the CD4 depletion effect in young versus older children, unpaired Mann-Whitney U tests (two-sided) were performed. Welch's t-tests (two-sided) were used to compare the somatic hypermutation levels between culture conditions and the clone size and diversity of inferred HA-specific and nonspecific B cell lineages. Two-sided, paired t-tests were used to compare the effect of PE stimulation with and without adjuvants on the frequency of PE+ B cells.

Next, IgH sequencing was performed to analyze the BCR repertoire in naive versus affinity matured B cells in response to LAIV stimulation. Here, cultures were prepared where naive, HA− B cells were the only B cell source (FIG. 4A). Day-0 HA+ B cells were used as the reference for high-affinity BCRs and to infer HA specificity. Organoid cultures were harvested on day 7 for bulk BCR repertoire sequencing (FIG. 4A). The repertoires of these cultures were compared to both the in vivo high-affinity HA+ B cells from day-0 tonsil cells that were depleted by FACS and the HA− naive B cells that went into culture preparations. Of the six donors tested, five secreted antibodies specific for vaccine components and four had detectable antibodies specific for the A/California 2009 H1N1 HA protein on day 7 after stimulation (FIG. 4B), showing that HA-specific antibodies can be derived from naive B cells.

Somatic hypermutation was significantly increased in day-7 LAIV-stimulated cultures compared to unstimulated control cultures and was particularly enhanced where A/California 2009 H1N1 HA antibodies were detected (IMD006, 013, 014 and 102; FIG. 4C). The development of BCR sequences likely (within one amino acid) to be specific for influenza strain A/California H1N1 were followed. In two of the six donors tested (IMD006 and IMD014), the B cell clones with inferred HA specificity for this strain were also larger than those without known HA specificity and this was dependent on vaccine stimulation (FIG. 4D). In these same donors, the average diversity was also increased, with more unique molecules per clonal lineage in HA-specific B cells from LAIV-stimulated cultures (FIG. 4d). Other expanded and diverse lineages only in the vaccine-stimulated cultures were found, likely responding to other antigens or strains. Direct evidence for BCR sequence evolution to increased HA affinity (FIG. 4E) was found. Here a root sequence with four nucleotide mutations from the germline heavy-chain sequence acquired an additional ten mutations to achieve an exact amino acid match to a known high-affinity HA specificity. Taken together, these data confirm that antigen-driven somatic hypermutation, affinity maturation and class switching are support in tonsil organoid cultures.

### Example 6: Contributions of Individual Cell Types to Influenza Responses

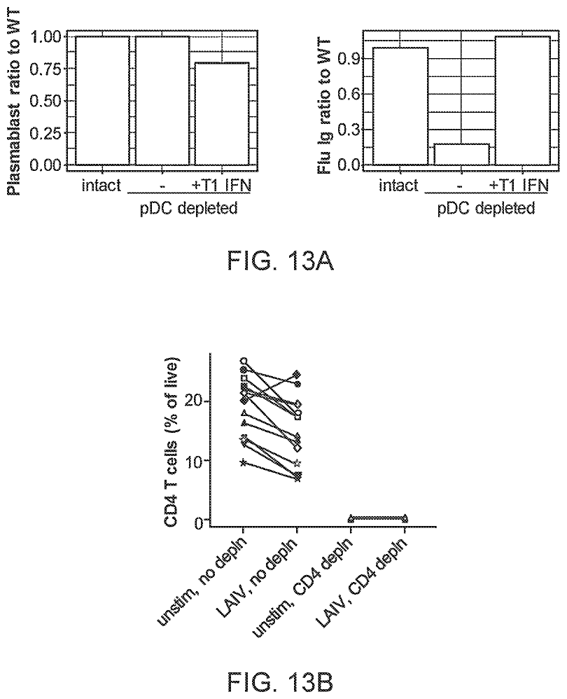

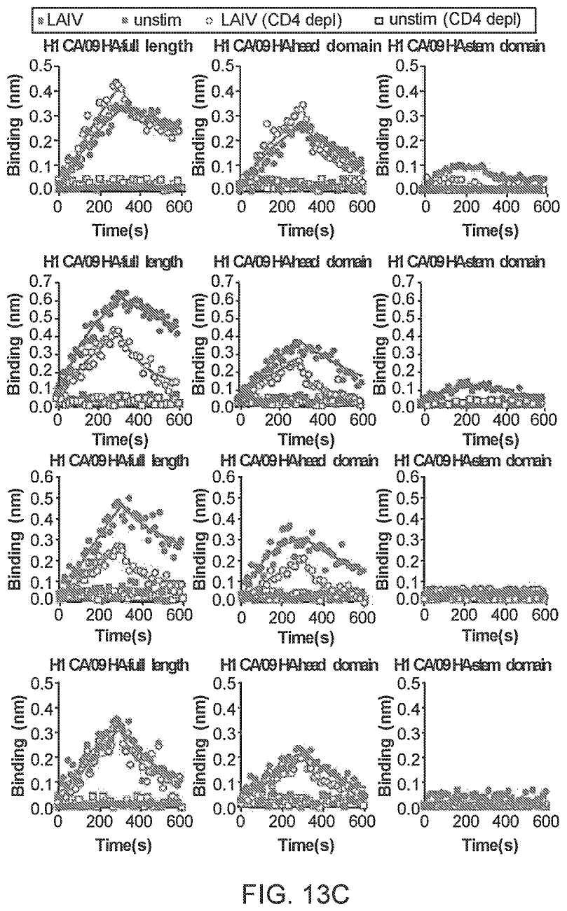

A major advantage of in vitro systems is the ability to define the essential components. Therefore, APC, T cell and B cell subsets were depleted and plasmablast differentiation was compared (FIGS. 5B and 5D) to wild type organoids. Despite their low starting frequency (0.1-0.5% of live), depleting myeloid and plasmacytoid dendritic cell (pDC) populations completely abrogated plasmablast differentiation (FIG. 5A) and depleting pDCs alone was sufficient to dramatically reduce the antibody response to LAIV (FIG. 5B). The effect of pDC loss was rescued by adding type I interferon (IFN), showing that type I IFN production is the main pDC function (FIG. 13A).

As expected, depleting CD4+ cells (FIG. 13B) significantly reduced plasmablast differentiation, although some influenza-specific antibodies were still detectable (FIGS. 5C-5D). The effect of donor age was analyzed and it was found that CD4+ T cell-depleted organoids from younger children were almost entirely unable to secrete influenza-specific antibody (FIG. 5C), in contrast to older donors who could make antibody responses.

The affinities of antibodies was characterized against A/California/07/2009 H1N1 HA in CD4+-depleted cultures. The dissociation rates for antibodies raised in the absence of CD4+ cells were four-to ten-fold higher than wild-type controls (FIG. 5F and FIG. 13C), even when the quantity of specific antibody was comparable between intact and CD4+-depleted cultures. Thus, CD4+ T cells are important for both the magnitude and affinity of the antibody response.

It was determined that three to four cell types were minimally required to consistently achieve detectable plasmablast differentiation and naive antibody responses against LAIV: naive B cells, APCs, CD4+ T cells and, in some donors, a mixed population of CD45 − stromal cells (FIG. 5G). Notably, naive CD4+ T cells were either as good or better than memory CD4+ T cells in their ability to help B cells differentiate into plasmablasts and stimulate influenza-specific antibodies (FIG. 5H).

### Example 7: Response to Other Antigens and the Effect of Adjuvants

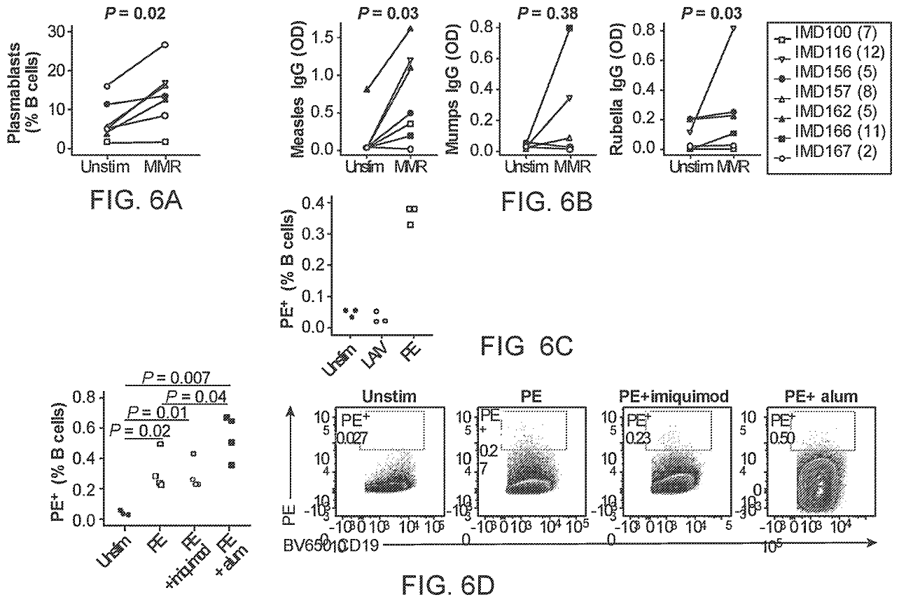

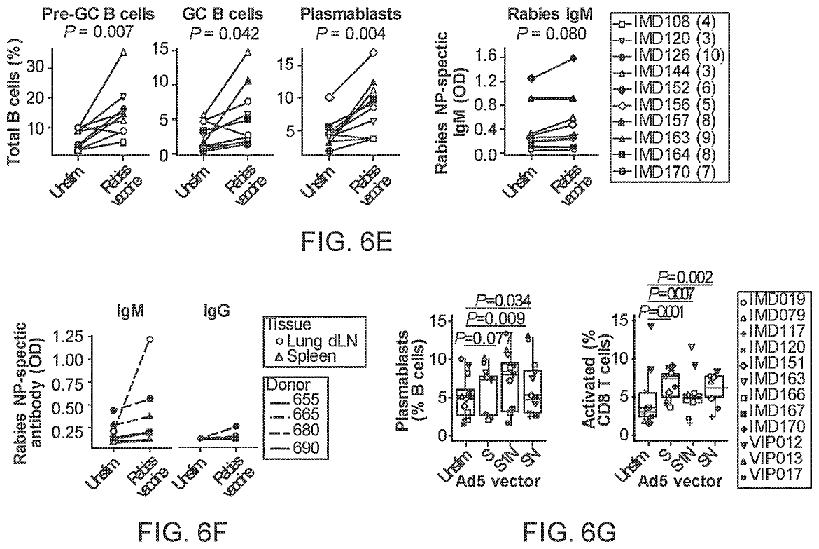

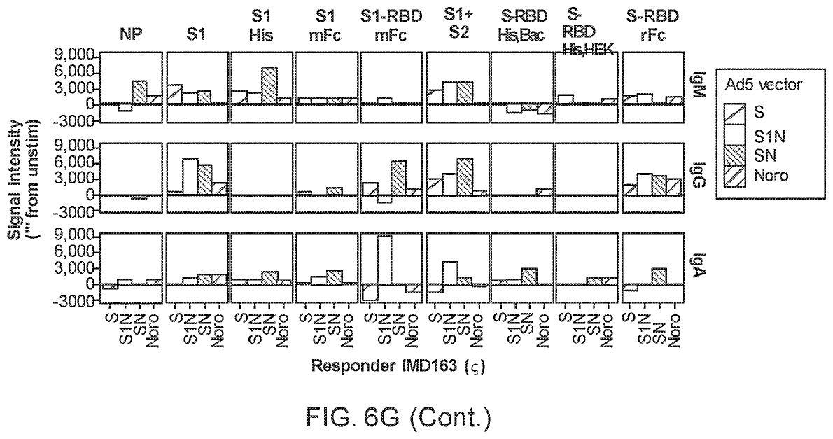

It was then investigated whether tonsil organoids could also respond to non-influenza memory antigens. Tonsil organoids were stimulated with the measles, mumps and rubella (MMR) vaccine, which is recommended to be administered at 12 months, and assessed plasmablast formation and antigen-specific IgG responses. These cultures had significantly increased plasmablasts compared to controls (FIG. 6A), and six of the seven donors made measles-specific IgG, while the mumps and rubella responses were weaker and only detected in three of the older children tested (FIG. 6B).

Affinity binding experiments. Binding affinities of the indicated antigens to antibody-containing supernatant samples were determined by biolayer interferometry using an Octet QK instrument (Pall ForteBio). For analysis of the effects of T cells on antibody responses, antibodies were purified from culture supernatants using a Protein G affinity column (GE Healthcare Life Sciences). The full-length, head and stem domain H1 CA/09 HA antigens were purified as described previously using a Ni-NTA affinity column followed by size-exclusion cleanup. The purified antigens were captured on anti-penta-his (HIS1K) biosensor tips in PBS-T (PBS with 0.05% Tween 20 (pH 7.4)). The ligand-bound sensors were dipped into control wells or purified antibodies (200-500 nM). A similar antibody concentration was used for all the evaluated conditions from an individual donor. Unliganded sensors dipped into the analyte served as controls for nonspecific binding. The traces were processed using ForteBio Data Analysis Software (v8.0). The data were fitted globally to a simple 1:1 Langmuir interaction model to obtain the kinetic parameters. Each binding interaction was repeated at least thrice.

To determine whether tonsil organoids could serve as a platform for priming antigen-specific adaptive responses, cells were first stimulated with naive antigen phycoerythrin (PE), with and without adjuvants. Here a substantial (tenfold) increase in PE+ B cells in PE-stimulated cultures was seen as compared to unstimulated or irrelevant antigen-stimulated cultures (FIG. 6C). The frequency of PE+ B cells could also be enhanced by alum hydrogel but not imiquimod (FIG. 6D), indicating that at least some naive responses can be elicited and modulated by appropriate adjuvants. The ability of tonsils, lung-draining lymph nodes and spleen cultures to respond to the T cell-dependent rabies vaccine was then tested. The donors had not previously received this vaccine. Tonsil B cells were more differentiated in vaccine-stimulated cultures after 14 d in organoid culture and four of ten donors had detectable increases in rabies nucleoprotein-specific IgM (FIG. 6E). Lung-draining lymph node and spleen organoids also showed modest rabies-specific antibody production, with some specific IgM but not IgG production, consistent with a priming response (FIG. 6F).

### Example 8: Testing Vaccine Candidates on Tonsil Organoids

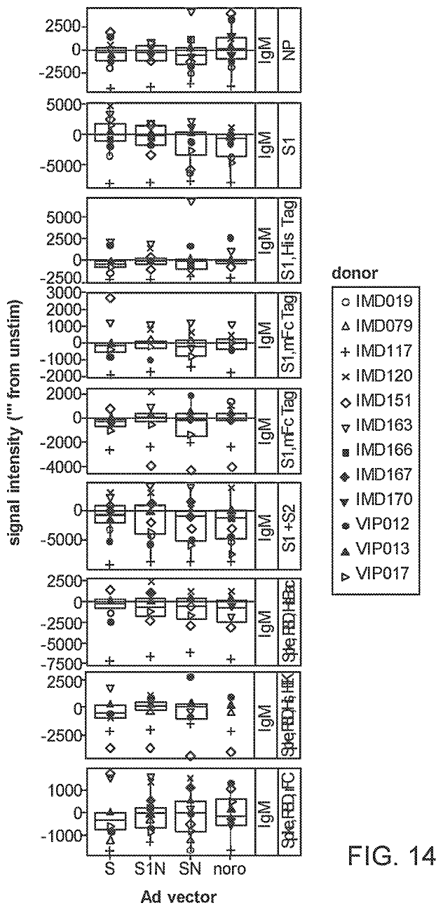

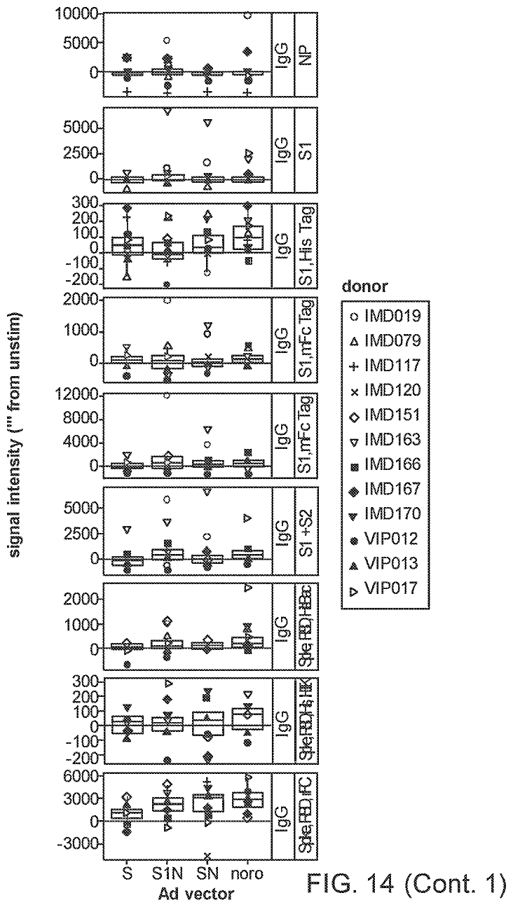

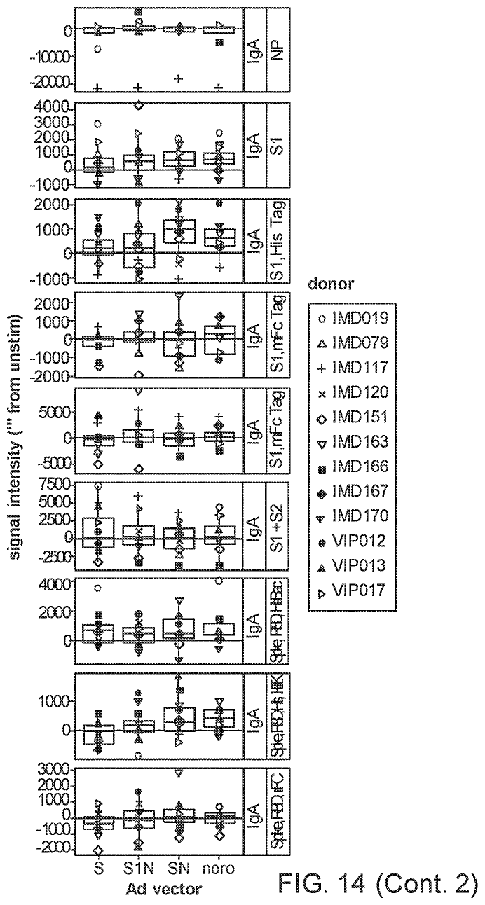

The ability of tonsil organoids to respond to a series of vaccine candidates developed in response to severe acute respiratory syndrome coronavirus 2 (SARS-COV-2) was investigated. These vaccine candidates use a replication-deficient type 5 adenovirus (Ad5) that encodes either the full-length viral spike protein, spike and nucleocapsid protein or the S1 spike subunit with nucleocapsid in the Ad5 E1 region. All tonsil tissue samples were collected before the SARS-COV-2 pandemic and thus naive to these antigens. On day 14 after stimulation, plasmablast differentiation in a subset of donors and significant CD8 T cell activation was observed compared to unstimulated controls (FIG. 6G). Using a protein microarray to detect antibody specificities against SARS-COV-2, specific IgG and IgA antibodies were present for some spike and nucleocapsid proteins in a few donors. One particularly robust responder, IMD163, is shown in FIG. 6G (data from all donors are available in FIG. 14). This shows that immune organoid cultures can be used to analyze and compare vaccine candidates to new and previously encountered antigens

### Example 9: Autoantibodies Generated From FOXP3-KO T Cells in Tonsil Organoids.

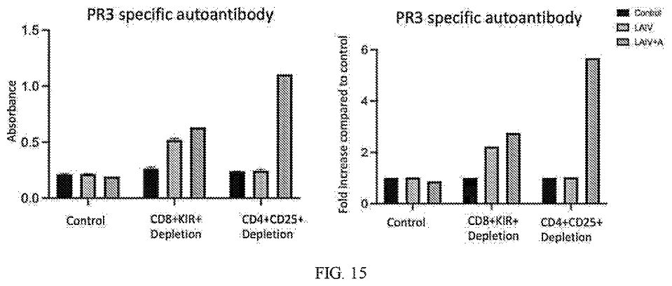

To identify which population of T cells play a main role in autobody generation, certain population of T cells was depleted from tonsil organoids and then stimulated with either LAIV or LAIV with self-antigen cocktails (LAIV+A). Self-antigen cocktails comprise proteinase 3 (PR3), double stranded DNA (dsDNA), histone, and SNRNP70, and these proteins are self-antigens commonly found in patient with autoimmune diseases (e.g., Lupus). The secretion of autoantibody specific to PR3 self-antigen was then evaluated by ELISA (FIG. 15). FIG. 15 shows that CD4+CD25+ T cells, which are a subset of regulatory T cells, play a suppressive role in autoantibody production. By depletion of CD4+CD25+ T cells, the tonsil organoids can produce a large amount of autoantibody specific to PR3 when compared to other conditions and other cell depletion.

The immune system carefully regulates T and B cell reactivities against self-antigen responses while focusing responses against foreign antigens. In this way, the body avoids autoreactivity for the most part, although this can break down in individuals with autoimmune diseases such as Lupus Erythematosus, Multiple Sclerosis, Rheumatoid Arthritis and dozens of other diseases where the immune system attacks a particular organoid or tissue. An important factor in avoiding self-reactivity are T cells expressing the transcription factor FoxP3, known as regulatory T cells. Because of the important role they play in preventing self-reactivity, mice and humans lacking FoxP3, for example, die in infancy from massive inflammation due to a lack of these T cells.

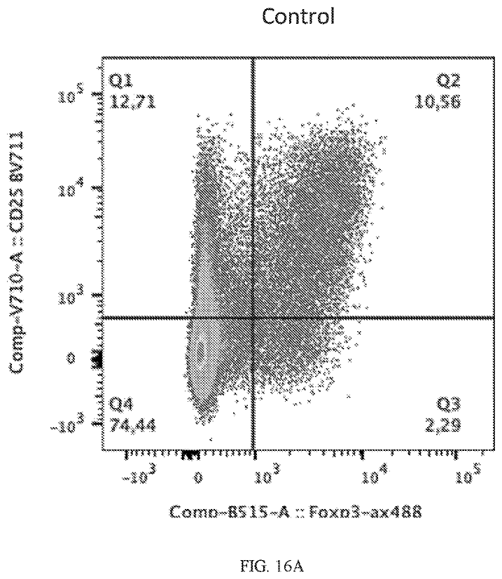

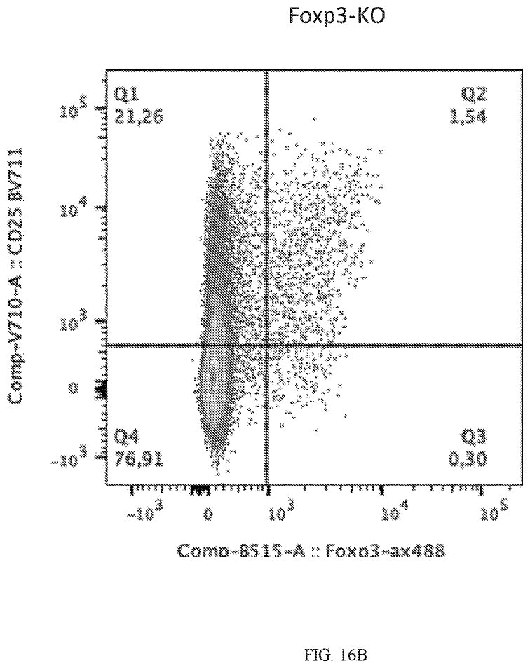

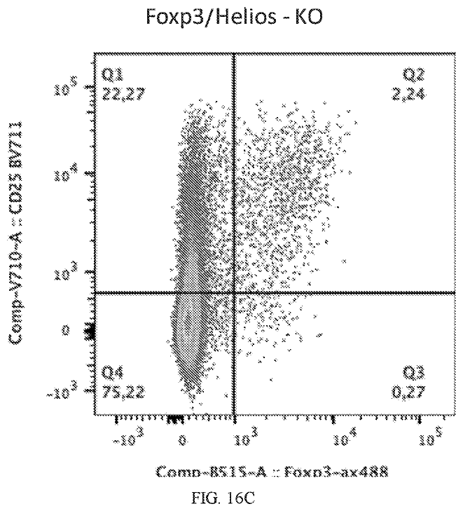

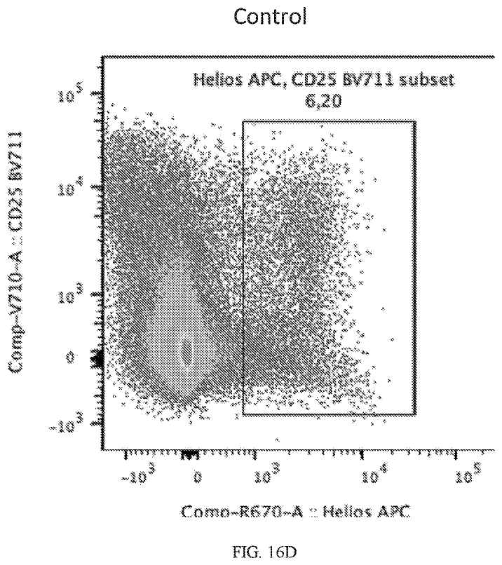

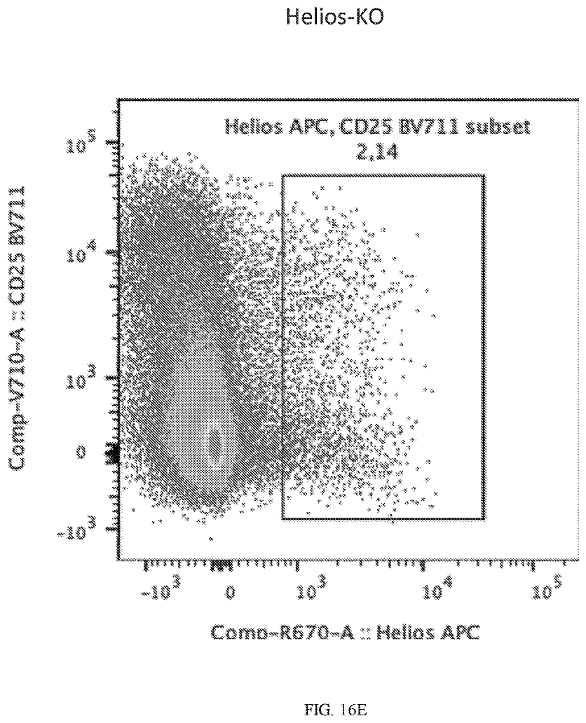

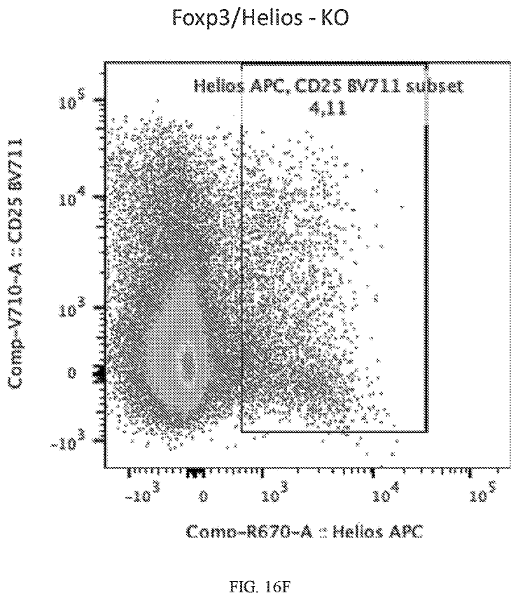

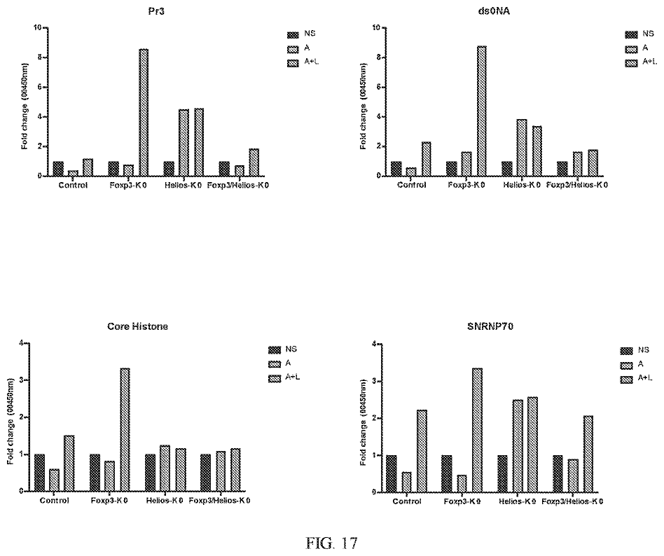

To investigate the FOXP3 function in T cells, first, T cells were isolated from tonsil organoid using Pan T cell isolation Kit (Miltenyi Biotec), and procedures were followed according to manufactures protocol. Next, CRISPR/Cas9 gene editing kit (Lonza) was used to perform FOXP3 knockout. Lonza P3 electroporation buffer was left to reach room temperature. Tonsil media was warmed to 37° C., and a culture plate with some media was also prewarmed. HIFI-Cas9 was diluted to 40 μM in Lonza P3 electroporation buffer. Ribonucleoprotein (RNP) complex was prepared by slowly mixed 1:1 volume of 40 μM FOXP3 gRNA and 40 μM HIFI-Cas9 to generate 20 μM Cas9-RNP (gRNA-Cas9 complex). The RNP complex was incubated at 37° C. for 15 minutes. If the experiment was performed at 0.1-1 million cells, 16-well strips were used by resuspending 3.5 μL/well of RNP complex in 20 μL/well of Lonza P3 electroporation buffer. Next, 21 μL of mixture was transferred to electroporation wells, and the 4D Nucleofector System (Lonza) was run using the manufactures electroporation protocol for unstimulated primary T cells. Immediately after electroporation, 80 μL of warm tonsil media was added to 16-well strip, and the cells were incubated for 30 minutes at 37 C in 5% CO₂ incubator in the electroporation plate before transferring the cells to a prewarmed culture plate. If the experiment was performed at 1-20 million cells, cuvettes were used by resuspending 17.5 μL/well of RNP complex in 100 μL/well of Lonza P3 electroporation buffer. Next, 115 μL of mixture was transferred to electroporation cuvettes, and the 4D Nucleofector System (Lonza) was run using the manufactures electroporation protocol for unstimulated primary T cells. Immediately after electroporation, 900 μL of warm tonsil media was added to the cuvettes, and the cells were incubated for 30 minutes at 37 C in 5% CO2 incubator in the electroporation plate before transferring the cells to a prewarmed culture plate. The result of FOXP3 KO in T cells was confirmed using FACS (data from FIGS. 16A-16F). These KO cells were cultured and stimulated with either LAIV or LAIV with self-antigen cocktails (LAIV+A). The secretion of autoantibodies to each self-antigen in the cocktails was measured using ELISA (data from FIG. 17). The result shows that upon the stimulation of LAIV+A, B cells interacting with FOXP3-KO T cells secrete a large amount of autoantibodies specific to each self-antigens including PR3, dsDNA, histone, and SNRNP70.

Results demonstrate that by eliminating FOXP3 gene expression via CRISPR in T cells in tonsil organoids in the invention disclosed herein, B cells are able to make autoantibodies that can mature, and secrete these antibodies. This does not happen when the FOXP3 gene is intact and functional. This result suggests that by the simple elimination of FOXP3 gene, the immune organoid system can be utilized to produce fully human antibodies to any antigen, self or non-self. Regarding antibody affinity, there is a natural limitation that typically falls in the low nanomolar range (1-10 nM). However, individuals with deficiencies in the AIRE gene, which is an autoimmune regulator gene, antibody affinity can be in the femtomolar range, which is much higher than normal. This suggests that the affinity of the antibody generated from the invention disclosed here is much greater than normal antibody. Results here indicate that this is also an aspect that is held in check by T cell regulation through FoxP3.

### Example 10. Identification of Tonsillar and Circulating CD4+ and CD8+ Regulatory T Cell Subsets in Donor-Matched Samples

FOXP3+ CD4+ regulatory T (Treg) cells and CD8+KIR+ T cells are critical in maintaining immune tolerance. In order to determine the percentage of two Treg subsets in tonsils and find out whether the percentage in tonsils is similar to the blood, two regulatory T cell subsets were compared in matched peripheral blood and tonsil samples from 7 adult donors.

### Human Studies

Ethics approval was granted by the Stanford University IRB. For children volunteers (IRB protocol 30837), written informed consent was obtained from the legal guardians of children aged 0-17 years and was also obtained from children aged 7 years and older. For adult volunteers (IRB protocol 60741), written informed consent was obtained and 20 ml of blood will be drawn from each patient. Subjects who are taking systemic immunomodulatory drugs, with a history of immunosuppressive or autoimmune diseases, or have a serious active infection at the time of the procedure will be excluded.

### Sample Collection and Processing

Tonsil samples were collected and processed as previously described (PMID: 33432170). Briefly, tonsils were removed by surgery for a spectrum of clinical presentations (e.g. otolaryngology patients undergoing tonsillectomy for sleep apnea and/or cardiothoracic patients undergoing thymectomy, etc). All surgery procedures are followed according to the Stanford University Institutional Review Board (IRB). Whole tonsils were collected in saline after surgery and decontaminated by immersed in an antimicrobial bath of Ham's F12 medium (Gibco) containing Normocin (InvivoGen), penicillin and streptomycin for 1 h at 4° C. Tonsil tissue was cut into small pieces (roughly 5 mm thickness) with scalpels and scissors and manually disrupted into a single cell suspension by processing through a 100-μm strainer with a syringe plunger. After washing with complete medium (RPMI with glutamax, 10% FBS, 1× nonessential amino acids, 1× sodium pyruvate, 1× penicillin-streptomycin, 1× Normocin (InvivoGen) and 1× insulin/selenium/transferrin cocktail (Gibco)), cells were frozen into aliquots in FBS+10% DMSO and stored at −140°° C. until use.

### Flow Cytometry

Culture organoids were resuspended by rinsing the membrane with media and collected from the transwells. Cells were washed with FACS buffer (PBS+0.1% BSA, 0.05% sodium azide and 2 mM EDTA) and stained at 4° C. with Fc block (1/20), live/dead Aqua Zombie stain (1/100), and anti-human antibodies. For FOXP3 staining, after surface staining, the cells were fixed and permeabilized (eBioscience) and stained intracellularly with anti-FOXP3. All analyzer data were collected on BD LSRII instruments and analyzed using FlowJo (TreeStar).

Multi-parameter flow cytometry-based characterization of CD4+ and CD8+ T cell population in peripheral blood mononuclear cells (PBMCs) and mononuclear cells from tonsils was performed (FIG. 19A). In tonsils, the frequency of total CD4+ Tregs and CD4+ Tregs expressing CXCR5 are significantly higher compared to the blood (FIG. 19A). Circulating CD4+ Tregs usually express a high surface level of CD25, which is served as an additional marker. However, almost half the population of tonsillar CD4+ Tregs cells express a low level of CD25, and the percentage of CD25low FOXP3+ CD4+ T cells is significantly higher compared to the blood (FIG. 19B). The percentage of CD8+ T cells expression KIR is comparable between tonsil and blood.

As shown in FIG. 19A, CD4⁺ T cells from tonsils and PBMCs were analyzed by FACS for CD25, FOXP3, and CXCR5 expression. The percentage of FOXP3⁺ cells, Tfr (CXCR5⁺FOXP3⁺), CD25+FOXP3⁺ cells in CD4⁺ T cells, and KIR+CD8+ T cells from donors (N=7) with PBMC and tonsil samples is shown in FIG. 19B.

### Example 11: Inflammatory and Autoimmune Cells in Lymphoid Cell Cultures With T Cells Genetically Modified to Lack FoxP3, or Granzyme B (GZMB) Expression

Next, in order to investigate the contribution of ablating FOXP3 and GZMB genes in T cells to the phenotype of the whole tonsil organoids, knockout experiments were performed.

### Cas9 Ribonucleoprotein (RNP) Assembly and Electroporation

Cas9 RNPs were prepared immediately before experiments by incubating 20 μM Cas9 with 20 μM sgRNA at 1:1 ratio at 37° C. for 15 min to a final concentration of 10 μM. T cells were electroporated with a Neon transfection kit and device (Invitrogen) as manufacture instruction. Briefly, T cells were gently resuspended in P3 buffer with supplement (Lonza Bioscience) at 2 million cells per 20 μl. The Cas9 RNPs and T cells were then mixed gently in P3 buffer. This mixture was then transferred to a 4D-Nucleofector cuvette (Lonza Bioscience) and pulsed with code EH105. After electroporation, the 4D-Nucleofector cuvette was placed in a 37° C. tissue culture incubator for 30 min to allow for cell recovery. After recovery, cells were ready for culture.

### Cell Culture

For the culture of cryopreserved cells, aliquots were thawed into a complete medium, enumerated, and resuspended to 6×10⁷ cells per ml for larger cultures or 2×10⁷ cells per ml for smaller cultures. Cells were plated, 100 μl per well, into permeable (0.4-μm pore size) membranes (24-well size PTFE or polycarbonate membranes in standard 12-well plates or 96-well polycarbonate membrane plates with single-well receiver trays; Corning or Millipore), with the lower chamber consisting of complete medium (1 ml for 12-well plates, 200 μl for 96-well plates) supplemented with 1 μg ml−1 of recombinant human B cell-activating factor (BAFF; BioLegend) and 1 ng/ml IL-21.

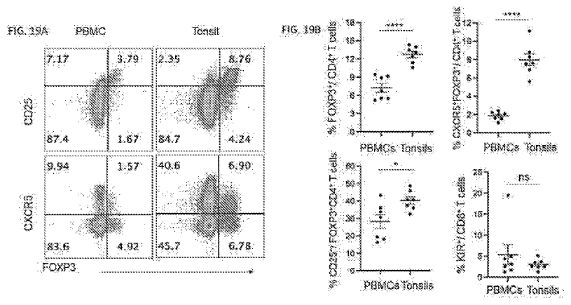

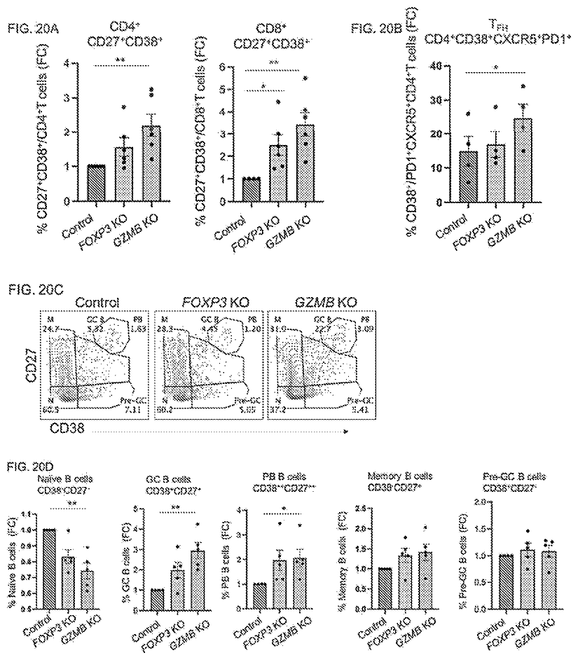

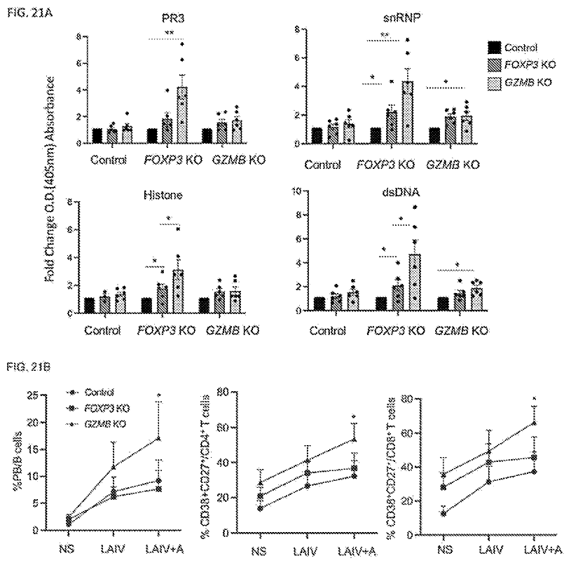

Shown in FIG. 20A are the fold change frequency of activated CD4, and CD8 T cells (CD27⁺CD38⁺) in FOXP3 KO and GZMB KO tonsil organoids over control, compared after 10-day culture. FIG. 20B shows the frequency of activated T_{FH} cells in control, FOXP3 KO, and GZMB KO tonsil organoids were compared after 10-day culture. Representative FACS staining of B cell differentiation phenotypes in control, FOXP3 KO, and GZMB KO tonsil organoids after 10-day culture is shown in FIG. 20C.

After 10-day culture, FOXP3 and GZMB KO tonsil organoids exhibited inflammatory changes characterized by an increased level of activated CD4+ and CD8+ expressing activation marker CD38 and co-stimulation molecule CD27 (FIG. 20A), and CD38+ T follicular helper (T_{FH}) cells (FIG. 20B) compared to control. In addition, KO tonsil organoids showed B cell activation as a reduced percentage of naïve B cells and an increased percentage of germinal center B cells and plasmablast B cells compared to control (FIGS. 20C and 20D). It should be noted that, compared to FOXP3 KO tonsil organoids, GZMB KO tonsil organoids showed a significantly higher level of inflammation with more T and B cells activation (FIG. 20D). However, no increased autoantibody production was detected in FOXP3 or GZMB KO tonsil organoids compared to control.

### Example 12: FOXP3 KO and GZMB KO Tonsil Organoids Showed Differential Autoimmune Phenotypes After Stimulation With LAIV and Autoantigen Stimulation

Viral infection has been suggested as a primary factor in the initiation of autoimmune diseases, and the mice model also developed an autoimmune phenotype upon viral infection. In order to determine whether the autoimmune response in the germinal center can be induced by viral antigens, after plating into transwells, the control, FOXP3 KO, or GZMB KO tonsil organoids were left unstimulated (NS) or stimulated with live attenuated influenza virus (LAIV). Additionally, LAIV and autoantigens cocktail (LAIV+A) were tested to determine if this could further increase autoantibodies. There was proteinase 3 (PR3), core histones, double-stranded DNA (dsDNA), and small nuclear ribonucleoproteins (snRNP) in the autoantigen cocktails, which are relatively common autoantigens targeted by autoantibodies found in patients with autoimmune diseases and viral infections.

### Cell Culture

Cell culture was performed as described in the previous examples. LAIV (1 μl per well, equivalent of 1.6×10⁴ to 1.6×10⁵ fluorescent focus units per strain; FluMist Quadrivalent, Medimmune) was then added directly to the cell-containing portion of the culture setup. Cultures were incubated at 37° C., 5% CO₂ with humidity, and supplemented with additional medium to the lower wells as necessary.

### Antibody Detection by ELISA

For detection of influenza-specific antibodies, ELISA plates (Costar) were coated with 0.1 μg per well of 2021-2022 Fluzone Quadrivalent inactivated influenza vaccine (Sanofi). For detection of autoantigen-specific antibodies, ELISA plates (Costar) were coated with 0.1 μg proteinase 3 (PR3), small nuclear ribonuclear protein (SnRNP), core histone, and dsDNA per well of as the capture antigen, respectively. Plates were coated with capture antigen overnight, followed by blocking reagents for two hours. Then cell supernatants from tonsil culture were added to coated, blocked plates. After washing with washing buffer, Horseradish peroxidase-conjugated anti-human secondary antibodies to IgM/IgG/IgA (Sigma) were added to the plate for 1 hour followed by TMB substrate solution (Thermo Scientific). Sulfuric acid was added to stop the reaction and the plates were read at 450 nm.

As shown in FIGS. 21A-21B, FOXP3 KO and GZMB KO tonsil organoids developed differential autoimmune phenotypes after stimulation with LAIV and autoantigen stimulation. FIG. 21A shows the fold change of the amount of autoantibody specific to Proteinase 3 (PR3), double-stranded DNA (dsDNA), small nuclear ribonucleoprotein 70 kDa (snRNP70), and core histone from control, FOXOP3 KO, and GzmB KO tonsil organoids. FIG. 21B shows the percentage of PB cells (CD27⁺⁺CD38⁺⁺ B cells), activated CD4⁺ and CD8⁺ T cells (CD38⁺CD27⁺) from non-stimulated (NS), LAIV stimulation, LAIV plus autoantigens (LAIV+A) stimulation from control, FOXP3 KO, GZMB KO tonsil organoids after 10-day culture. It is important to note that although GZMB KO or CD8+KIR+ T cell-depleting tonsil organoids generated mild amounts of autoantibody upon stimulation, they had a significantly higher percentage of activated T cells and plasmablasts than FOXP3 KO and control (FIG. 21B).

### Example 13: FOXP3 KO Tonsil Organoids Stimulated With LAIV Generated High-Affinity HA Antibodies

Since FOXP3 is critical in regulating autoantibody response, to determine whether FOXP3 could also affect antigen-specific antibody response including the quality of the antibodies, the binding affinity of antigen-specific antibodies produced from LAIV-stimulated FOXP3 KO tonsil organoids was measured. Antibodies against hemagglutinin (H1), which is the primary target of antibodies in vaccinated individuals were focused.

### Binding Affinity Measurement

The binding affinity of full-length H1 California/04/2009 influenza hemagglutinin (CA/09 HA) to the antibodies secreted into the culture media of the indicated human tonsil organoids on day 7 after LAIV stimulation was measured using biolayer interferometry (BLI) using an Octet QK instrument (Pall ForteBio, CA, USA). The antigen (H1 CA/09 HA) diluted in PBST (PBS with 0.05% Tween 20, pH 7.4) was captured using Ni-NTA biosensors. The ligand-bound biosensors were dipped into a serially diluted culture supernatant. The association and dissociation were both monitored for 1 h. Double referencing was performed using unliganded biosensors and an irrelevant E. coli maltose binding protein (MBP). The dissociation rate constant (K_{d}) was determined by a global fit of exponential decay kinetics. Each binding interaction was performed in duplicate.

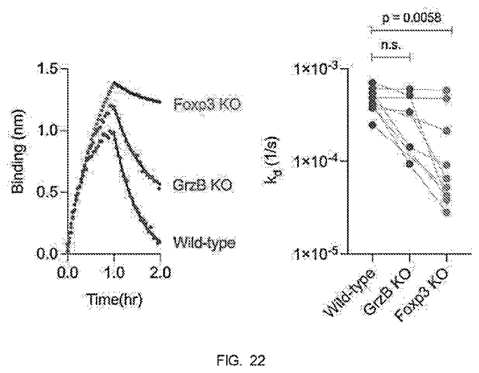

Live attenuated flu vaccine (LAIV) was used as an adjuvant in the FoxP3 and Granzyme B KOs, allowing measurement of the resulting bulk anti influenza HA affinities using interferometry. Shown in FIG. 22, there was a marked increase in the affinity in most donors (n=9) with the FoxP3 KO, up to an 18-fold decrease in the off rate (k_{d}), with a more modest effect of the GrzB KO in a few donors.

The dissociation rate constant (Kd) for antibodies in the FOXP3 KO condition to H1 CA/09 HA was on average 4-fold lower in comparison to donor-matched wild-type condition. Results in FIG. 22 show that breaching the regulatory checkpoint by knocking-out FOXP3 can generate high-affinity HA antibodies, suggesting an efficient strategy to manipulate the binding affinity of antigen-specific antibodies by using genetically modified tonsil systems.

Overall, these results show that that FOXP3 KO in tonsil T cells produced autoantibodies when stimulated with a panel of classical autoantigens, whereas GZMB KO showed a marked increase in autoreactive CD8+ T cells and plasmablasts, but only a low level of autoantibodies. CD8+ and CD4+ Tregs have distinct and complementary roles in regulating cellular and humoral responses and preventing autoimmunity. Importantly, this genetically modified tonsil system allows in vitro modeling of autoreactive immune cell responses, which remains a key obstacle to understanding mechanisms of autoimmunity in humans. These results also show an increase in autoantibody production in FOXP3 KO with defective CD4+ Tregs, and an increase in follicular helper T cells and autoreactive CD8+ T cells in GZMB KO with defective CD8+ Tregs.

While embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. An in vitro cell cluster comprising lymphoid cells,
wherein said in vitro cell cluster derived from lymphoid tissue of a subject, said in vitro cell cluster comprising a germinal center configured to produce antibodies to an antigen, wherein said germinal center comprises genetically modified T-cells.

2. The in vitro cell cluster of claim 1, wherein said antigen is a self-antigen.

3. The in vitro cell cluster of claim 1, wherein said antigen is a polysaccharide, lipid, nucleic acid, peptide, protein or fragment thereof.

4. The in vitro cell cluster of claim 1, wherein said protein is a viral protein, a growth factor, a cancer related protein, or an auto-immune disease related protein.

5. The in vitro cell cluster of any of claims 1-4, wherein said antigen is expressed by a tissue of said subject.

6. The in vitro cell cluster of claim 1, wherein said antigen is a vaccine or vaccine candidate.

7. The in vitro cell cluster of any of claims 1-6, wherein said genetically modified T-cells are regulatory T-cells.

8. The in vitro cell cluster of any of claims 1-7, wherein said genetically-modified T-cells are modified to knock down or knock out an expression of a forkhead box transcription factor.

9. The in vitro cell cluster of claim 8, wherein said forkhead box transcription factor is FoxP3.

10. The in vitro cell cluster of any of claims 1-9, wherein said genetically modified T-cells are CD8⁺ T-cells.

11. The in vitro cell cluster of claim 10, wherein said genetically-modified CD8+ T-cells are modified to knock down or knock out an expression of granzyme B.

12. The in vitro cell cluster of any of claims 1-11, further comprising one or more adjuvants.

13. The in vitro cell cluster of claim 12, wherein said one or more adjuvants comprise aluminum hydroxide or imiquimod.

14. The in vitro cell cluster of any of claims 1-13, wherein said germinal center is configured to perform one or more of: hypermutation maturation, affinity maturation, plasmablast differentiation, and class switching recombination.

15. The in vitro cell cluster of any of claims 1-14, wherein said cells are configured to differentiate to form said in vitro cell cluster upon exposure to said antigen.

16. The in vitro cell cluster of any of claims 1-15, wherein said lymphoid cells are derived from tonsil tissue, spleen tissue, adenoid tissue, thymus tissue, or lymph node tissue.

17. The in vitro cell cluster of any of claims 1-16, wherein said germinal center comprises antigen presenting cells (APCs) and T-cells at least partially surrounding said functional germinal center.

18. in vitro cell cluster of claim 17, wherein said APCs comprise B-cells.

19. The in vitro cell cluster of claim 17, wherein said APCs comprise dendritic cells.

20. The in vitro cell cluster of claim 17, wherein said dendritic cells are follicular dendritic cells.

21. The in vitro cell cluster of any of claims 1-20, comprising CD38+ B-cells.

22. The in vitro cell cluster of any of claims 1-20, comprising CD27+ B-cells.

23. The in vitro cell cluster of any of claims 1-20, comprising CD8+ T-cells.

24. The in vitro cell cluster of any of claims 1-20, comprising CD4+ T-cells.

25. The in vitro cell cluster of any of claims 1-24, wherein said antibodies have an affinity to said antigen between 1 nanomolar and 10 femtomolar.

26. In in vitro cell culture of any of claims 1-25, wherein the lymphoid cells are human cells.

27. A method for generating antibodies from a lymphoid organoid, comprising:
(a) placing lymphoid cells in a media to produce said lymphoid organoid, wherein said lymphoid organoid comprises T-cells and B-cells, wherein said T-cells are modified to have a decreased regulation of B-cells as compared to unmodified T-cells;
(b) introducing an antigen to said media;
(c) incubating said lymphoid organoid with said antigen to generate said antibodies; and
(d) isolating said antibodies or nucleic acids encoding said antibodies from said lymphoid organoid.

28. The method of claim 27 wherein said lymphoid cells are obtained from a subject, wherein said antigen is expressed by a tissue of said subject.

29. The method of claim 28, wherein said subject is human.

30. The method of any of claims 27-29, wherein said T-cells are modified to reduce or eliminate expression of a forkhead box transcription factor.

31. The method of claim 30, wherein said forkhead box transcription factor is FoxP3.

32. The method of any of claims 27-31, wherein said T-cells are modified to reduce or eliminate expression of granzyme b (GZMB).

33. The method of any of claims 27-32, wherein (a) further comprises introducing a B-cell activating factor to said media.

34. The method of any of claims 27-33, wherein (b) further comprises introducing an adjuvant to said media.

35. The method of claim 34, wherein said adjuvant is aluminum hydroxide or imiquimod.

36. The method of claim 34 or 35, wherein (c) further comprises incubating said lymphoid organoid with said antigen and said adjuvant.

37. The method of any of claims 32-36, wherein said adjuvant is introduced after said antigen.

38. The method of any of claims 27-37, wherein said incubating is for at least 48 hours.

39. The method of any of claims 27-38, further comprise obtaining said lymphoid cells from a subject.

40. The method of claim 39, wherein said lymphoid cells are obtained by biopsy.

41. The method of any of claims 27-40, wherein said lymphoid cells are derived from tonsil tissue, spleen tissue, adenoid tissue, thymus tissue, or lymph node tissue.

42. The method of any of claims 27-41, wherein said antigen is a peptide, protein, or fragment thereof.

43. The method of any of claims 27-41, wherein said protein is a growth factor, a cancer related protein, or an auto-immune disease related protein.

44. The method of any of claims 27-41, wherein said antigen is a polysaccharides, lipid or nucleic acid.

45. The method of any of claims 27-41, wherein said antigen is a vaccine or vaccine candidate.

46. The method of any of claims 27-45, further comprising introducing an immune system stimulant.

47. The method of claim 46, wherein said immune system stimulant is a live attenuated influenza virus (LAIV).

48. The method of claim 47, wherein said LAIV is introduced with said antigen.

49. The method of any of claims 27-48, wherein said antibodies have an affinity to said antigen between 1 nanomolar and 10 femtomolar.

50. An in vitro cell cluster comprising lymphoid cells, wherein said in vitro cell cluster is derived from lymphoid tissue of a subject, said in vitro cell cluster comprising a germinal center configured to produce antibodies to a self-antigen, wherein said germinal center comprises genetically modified T-cells.

51. A method for generating antibodies from a lymphoid organoid, comprising:
(a) placing lymphoid cells in a media to produce said lymphoid organoid, wherein said lymphoid organoid comprises T-cells and B-cells, wherein said T-cells are modified to have a decreased regulation of B-cells as compared to unmodified T-cells;
(b) introducing a self-antigen to said media;
(c) incubating said lymphoid organoid with said self-antigen to generate said antibodies; and
(d) isolating said antibodies from said lymphoid organoid.
